(19) United States
(12) Patent Application Publication
(10) Pub. No.: US 2008/0255188 A1
(43) Pub. Date: Oct. 16, 2008

(54) Muscarinic Receptor Antagonists

(76) Inventors: Naresh Kumar, Gurgaon (IN); Mohammad Salman, Princeton, NJ (US); Kirandeep Kaur, Gurgaon (IN); Anita Chugh, New Delhi (IN); Sandeep Sinha, New Delhi (IN)

Correspondence Address: RANBAXY INC., 600 COLLEGE ROAD EAST, SUITE 2100, PRINCETON, NJ, 08540, US

(21) Appl. No.: 11/995,433

(22) PCT Filed: Jul. 11, 2006
(86) PCT No.: PCT/IB06/52349

§ 371 Date: Jun. 11, 2008

(30) Foreign Application Priority Data
Jul. 11, 2005 (IN) 1798/DEL/2005

(51) Int. Cl. A61K 31/472; C07D 217/26

(52) U.S. Cl. 514/307; 546/147

(57) ABSTRACT

This present invention generally relates to muscarinic receptor antagonists of Formula (I), which are useful, among other uses, for the treatment of various diseases of the respiratory, urinary and gastrointestinal systems mediated through muscarinic receptors. The invention also relates to the process for the preparation of disclosed compounds, pharmaceutical compositions containing the disclosed compounds, and the methods for treating diseases mediated through muscarinic receptors.

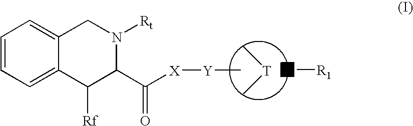

### FIELD OF THE INVENTION

This present invention generally relates to muscarinic receptor antagonists, which are useful, among other uses, for the treatment of various diseases of the respiratory, urinary and gastrointestinal systems mediated through muscarinic receptors. The invention also relates to the process for the preparation of disclosed compounds, pharmaceutical compositions containing the disclosed compounds, and the methods for treating diseases mediated through muscarinic receptors.

### BACKGROUND OF THE INVENTION

Physiological effects elicited by the neurotransmitter acetylcholine are mediated through its interaction with two major classes of acetylcholine receptors—the nicotinic and muscarinic acetylcholine receptors. Muscarinic receptors belong to the superfamily of G-protein coupled receptors and five molecularly distinct subtypes are known to exist (M₁, M₂, M₃, M₄ and M₅).

These receptors are widely distributed on multiple organs and tissues and are critical to the maintenance of central and peripheral cholinergic neurotransmission. The regional distribution of these receptor sub-types in the brain and other organs has been documented (for example, the M₁ subtype is located primarily in neuronal tissues such as cereberal cortex and autonomic ganglia, the M₂ subtype is present mainly in the heart and bladder smooth muscle, and the M₃ subtype is located predominantly on smooth muscle and salivary glands (Nature, 323, p. 411 (1986); Science, 237, p. 527 (1987)).

A review in Curr. Opin. Chem. Biol., 3, p. 426 (1999), as well as in Trends in Pharmacol. Sci., 22, p. 409 (2001) by Eglen et. al., describes the biological potentials of modulating muscarinic receptor subtypes by ligands in different disease conditions, such as Alzheimer's disease, pain, urinary disease condition, chronic obstructive pulmonary disease, and the like.

The pharmacological and medical aspects of the muscarinic class of acetylcholine agonists and antagonists are presented in a review in Molecules, 6, p. 142 (2001). Birdsall et al. in Trends in Pharmacol. Sci., 22, p. 215 (2001) has also summarized the recent developments on the role of different muscarinic receptor subtypes using different muscarinic receptor of knock out mice.

Almost all the smooth muscles express a mixed population of M₂ and M₃ receptors. Although the M₂-receptors are the predominant cholinoreceptors, the smaller population of M₃-receptors appears to be the most functionally important as they mediate the direct contraction of these smooth muscles. Muscarinic receptor antagonists are known to be useful for treating various medical conditions associated with improper smooth muscle function, such as overactive bladder syndrome, irritable bowel syndrome and chronic obstructive pulmonary disease. However the therapeutic utility of antimuscarinics has been limited by poor tolerability as a result of treatment related, frequent systemic adverse events such as dry mouth, constipation, blurred vision, headache, somnolence and tachycardia. Thus, there exists a need for novel muscarinic receptor antagonists that demonstrate target organ selectivity.

WO 04/005252 discloses azabicyclo derivatives described as musacrinic receptor antagonists. WO 04/004629, WO 04/052857, WO 04/067510, WO 04/014853, WO 04/014363 discloses 3,6-disubstituted azabicyclo[3.1.0]hexane derivatives described as useful muscarinic receptor antagonists. WO 04/056811 discloses flaxavate derivatives as muscarinic receptor antagonists. WO 04/056810 discloses xanthene derivatives as muscarinic receptor antagonists. WO 04/056767 discloses 1-substituted-3-pyrrolidine derivatives as muscarinic receptor antagonists. WO 04/089363, WO 04/089898, WO 04/069835, WO 04/089900 and WO 04/089364 discloses substituted azabicyclohexane derivatives as muscarinic receptor antagonists. WO 06/018708 disclose pyrrolidine derivatives as muscarinic receptor antagonists. WO 06/35303 discloses azabicyclo derivatives as muscarinic receptor antagonists.

J. Med. Chem., 44, p. 984 (2002), describes cyclohexylmethylpiperidinyl-triphenylpropioamide derivatives as selective M₃ antagonist discriminating against the other receptor subtypes. J. Med. Chem., 36, p. 610 (1993), describes the synthesis and antimuscarinic activity of some 1-cycloalkyl-1-hydroxy-1-phenyl-3-(4-substituted piperazinyl)-2-propanones and related compounds. J. Med. Chem., 34, p. 3065 (1991), describes analogues of oxybutynin, synthesis and antimuscarinic activity of some substituted 7-amino-1-hydroxy-5-heptyn-2-ones and related compounds. Bio-Organic Medicinal Chemistry Letters, 15, p. 2093 (2005) describes synthesis and activity of analogues of oxybutynin and tolterodine.

The present invention fills the need of muscarinic receptor antagonists useful in the treatment of disease states associated with improper smooth muscle function and respiratory disorders.

### SUMMARY OF THE INVENTION

In one aspect, there are provided muscarinic receptor antagonists, which can be useful as safe and effective therapeutic or prophylactic agents for the treatment of various diseases of the respiratory, urinary and gastrointestinal systems. Also provided are processes for synthesizing such compounds.

In another aspect, pharmaceutical compositions containing such compounds are provided together with acceptable carriers, excipients or diluents which can be useful for the treatment of various diseases of the respiratory, urinary and gastrointestinal systems. The enantiomers, diastereomers, N-oxides, polymorphs, pharmaceutically acceptable salts and pharmaceutically acceptable solvates of these compounds as well as metabolites having the same type of activity are also provided, as well as pharmaceutical compositions comprising the compounds, their metabolites, enantiomers, diastereomers, N-oxides, polymorphs, solvates or pharmaceutically acceptable salts thereof, in combination with a pharmaceutically acceptable carrier and optionally included excipients.

Other aspects will be set forth in the description which follows, and in part will be apparent from the description or may be learnt by the practice of the invention.

In accordance with one aspect, there are provided compounds having the structure of Formula I

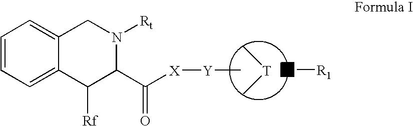

and their pharmaceutically acceptable salts, pharmaceutically acceptable solvates, enantiomers, diastereomers, polymorphs or N-oxides wherein

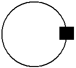

represents a nitrogen-containing ring having 5-8 ring atoms (for example, carbon atoms);
T is a bridging group selected from —(CH₂)ₙ—, —CH(Q)CH₂—, —CH₂CH(Q)CH₂—, —CH(Q)-, —CH₂—O—CH₂—, or —CH₂—NH—CH₂— (wherein the bridging group is attached to the two carbon atoms of the ring

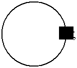

n is an integer selected from 0-3 (wherein when n is zero then T represents a direct bond);

Rf is hydrogen or Rₚ (defined below);

Y is alkylene or no atom (wherein when Y is no atom then X is directly attached to the ring

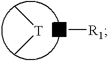

X is O, S or NRₛ (wherein Rₛ is as defined below);
R₁ is selected from hydrogen, aralkyl, halogen, —COOR₂ or Rᵤ (wherein Rᵤ is the same as defined below);
Rₜ is hydrogen, —COOR₂ or Rₚ (wherein Rₚ is alkyl, alkenyl, alkynyl, heterocyclylalkyl, heteroarylalkyl, aryl, aralkyl, heteroaryl, cycloalkyl or heterocyclyl);
Rᵤ is alkyl, aryl, heteroaryl, cycloalkyl, heterocyclyl, heteroarylalkyl, heterocyclylalkyl, —C(═O)NRₓR_{y}, —SO₂R₃, acyl (wherein R₃, Rₓ and R_{y} are the same as defined below);
Rₓ and R_{y} are independently selected from hydrogen, alkyl, cycloalkyl, aryl, halogen, aralkyl, heteroaryl, heterocyclyl, heteroarylalkyl or heterocyclylalkyl; Rₓ and R_{y} may also together join to form a heterocyclyl ring.
Rₛ is selected from hydrogen, alkyl, cycloalkyl, aryl, heteroaryl, heterocyclyl, heteroaryl, aralkyl, heteroarylalkyl or heterocyclylalkyl;
R₂ is independently selected from alkyl, alkenyl, alkynyl, aryl, aralkyl, heteroaryl, cycloalkyl, heterocyclyl, heterocyclylalkyl or heteroarylalkyl; and
R₃ is alkyl, aryl, heteroaryl, heterocyclyl, cycloalkyl, aralkyl, heteroarylalkyl, heterocyclylalkyl or —NRₓR_{y} (Rₓ and R_{y} are the same as defined earlier).

In accordance with a second aspect, there is provided a method for treatment or prophylaxis of an animal or a human suffering from a disease or disorder of the respiratory, urinary and gastrointestinal systems, wherein the disease or disorder is mediated through muscarinic receptors. The method includes administration of at least one compound having the structure of Formula I.

In accordance with a third aspect, there is provided a method for treatment or prophylaxis of an animal or a human suffering from a disease or disorder of the respiratory system such as bronchial asthma, chronic obstructive pulmonary disorders (COPD), pulmonary fibrosis, and the like; urinary system which induce such urinary disorders as urinary incontinence, lower urinary tract symptoms (LUTS), etc.; and gastrointestinal system such as irritable bowel syndrome, obesity, diabetes and gastrointestinal hyperkinesis with compounds as described above, wherein the disease or disorder is associated with muscarinic receptors.

In accordance with a fourth aspect, there are provided processes for preparing the compounds as described above.

The compounds described herein exhibit affinity for M₃ receptors, as determined by in vitro receptor binding assay Pharmaceutical compositions for the possible treatment for the disease or disorders associated with muscarinic receptors are provided. In addition, the compounds can be administered orally or parenterally.

The following definitions apply to terms as used herein:

The term “alkyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulphinyl, sulphonyl group or —NR_{α}—, wherein R_{α} can be hydrogen, alkyl, cycloalkyl, alkenyl, cycloalkenyl, alkynyl, aryl, acyl, aralkyl, —C(═O)OR_{λ}, SOₘR_{ψ} or —C(═O)NR_{λ}R_{π}. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl, heterocyclyl, heteroaryl, (heterocyclyl)alkyl, cycloalkoxy, —CH═N—O(C₁₋₆alkyl), —CH═N—NH(C₁₋₆alkyl), —CH═N—NH(C₁₋₆alkyl)-C₁₋₆alkyl, arylthio, thiol, alkylthio, aryloxy, nitro, aminosulfonyl, aminocarbonylamino, —NHC(═O)R_{λ}, —NR_{λ}R_{π}, —C(═O)NR_{λ}R_{π}, —NHC(═O)NR_{λ}R_{π}, —C(═O)heteroaryl, C(═O)heterocyclyl, —O—C(═O)NR_{λ}R_{π}, {wherein R_{λ} and R_{π} are independently selected from hydrogen, halogen, hydroxy, alkyl, alkenyl, alkynyl, alkenyl, alkoxy, cycloalkyl, cycloalkenyl, aryl, aralkyl, heterocyclyl, heteroaryl, heterocyclylalkyl, heteroarylalkyl or carboxy}, nitro or —SOₘR_{ψ} (wherein m is an integer from 0-2 and R_{ψ} is hydrogen, alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, alkenyl, alkynyl, carboxy, —NR_{λ}R_{π}, —C(═O)NR_{λ}R_{π}, —OC(═O)NR_{λ}R_{π}, —NHC(═O)NR_{λ}R_{π}, hydroxy, alkoxy, halogen, CF₃, cyano, and —SOₘR_{ψ}; or an alkyl group also may be interrupted by 1-5 atoms of groups independently selected from oxygen, sulfur or —NR_{α}— (wherein R_{α}, R_{λ}, R_{π}, m and R_{ψ} are the same as defined earlier). Unless otherwise constrained by the definition, all substituents may be substituted further by 1-3 substituents selected from alkyl, alkenyl, alkynyl, carboxy, carboxyalkyl, —NR_{λ}R_{π}, —C(═O)NR_{λ}R_{π}, —O—C(═O)NR_{λ}R_{π}, hydroxy, alkoxy, halogen, CF₃, cyano, and —SOₘR_{ψ} (wherein R_{λ}, R_{π}, m and R_{ψ} are the same as defined earlier); or an alkyl group as defined above that has both substituents as defined above and is also interrupted by 1-5 atoms or groups as defined above.

The term “alkylene,” as used herein, refers to a diradical branched or unbranched saturated hydrocarbon chain having from 1 to 6 carbon atoms and one or more hydrogen can optionally be substituted with alkyl, hydroxy, halogen or oximes. This term can be exemplified by groups such as methylene, ethylene, propylene isomers (e.g., —CH₂CH₂CH₂ and —CH(CH₃)CH₂) and the like. Alkylene may further be substituted with one or more substituents such as alkyl, alkenyl, alkynyl, alkoxy, cycloalkyl, acyl, acylamino, acyloxy, alkoxycarbonylamino, azido, cyano, halogen, hydroxy, oxo, thiocarbonyl, carboxy, arylthio, thiol, alkylthio, aryloxy, heteroaryloxy, aminosulfonyl, —COOR_{ψ}, —NHC(═O)R_{λ}, —NR_{λ}R_{π}, —C(═O)NR_{λ}R_{π}, —NHC(═O)NR_{λ}R_{π}, —C(═O)heteroaryl, C(═O)heterocyclyl, —O—C(═O)NR_{λ}R_{π}, nitro, —S(O)ₘR_{λ} (wherein R_{λ}, R_{π}, m and R_{ψ} are the same as defined earlier). Unless otherwise constrained by the definition, all substituents may be further substituted by 1-3 substituents chosen from alkyl, alkenyl, alkynyl, carboxy, —COOR_{ψ}, —NR_{λ}R_{π}, —C(═O)NR_{λ}R_{π}, —OC(═O)NR_{λ}R_{π}, —NHC(═O)NR_{λ}R_{π}, hydroxy, alkoxy, halogen, CF₃, cyano, and —S(O)ₘR_{ψ} (wherein R_{λ}, R_{π}, m and R_{ψ} are the same as defined earlier). Alkylene can also be optionally interrupted by 1-5 atoms of groups independently chosen from oxygen, sulfur and —NR_{α} (wherein R_{α} is the same as defined earlier). Unless otherwise constrained by the definition, all substituents may be further substituted by 1-3 substituents selected from hydrogen, alkyl, cycloalkyl, alkenyl, alkynyl, aryl, acyl, aralkyl, alkoxy, hydroxy, carboxy, —C(═O)OR_{ψ}, halogen, CF₃, cyano, —NR_{λ}R_{π}, —S(O)ₘR_{ψ}, —C(═O)NR_{λ}R_{π}, —OC(═O)NR_{λ}R_{π}, —CONH—, —C═O or —C═NOH (wherein R_{λ}, R_{π}, m and R_{ψ} are the same as defined earlier).

The term “alkenyl,” unless otherwise specified, refers to a monoradical of a branched or unbranched unsaturated hydrocarbon group having from 2 to 20 carbon atoms with cis, trans or geminal geometry. Alkenyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkenyl is attached to a heteroatom, the double bond cannot be alpha to the heteroatom. Alkenyl groups may be substituted further with one or more substituents selected from alkyl, alkenyl, alkynyl, alkoxy, cycloalkyl, cycloalkenyl, acyl, acylamino, acyloxy, —NHC(═O)R_{λ}, —NR_{λ}R_{π}, —C(═O)NR_{λ}R_{π}, —NHC(═O)NR_{λ}R_{π}, —O—C(═O)NR_{λ}R_{π}, alkoxycarbonylamino, azido, cyano, halogen, hydroxy, oxo, keto, carboxyalkyl, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, hydroxyamino, alkoxyamino, nitro or SOₘR_{ψ} (wherein R_{λ}, R_{π}, m and R_{ψ} are as defined earlier). Unless otherwise constrained by the definition, alkenyl substituents optionally may be substituted further by 1-3 substituents selected from alkyl, alkenyl, alkynyl, carboxy, hydroxy, alkoxy, halogen, —CF₃, cyano, —NR_{λ}R_{π}, —C(═O)NR_{λ}R_{π}, —O—C(═O)NR_{λ}R_{π}, and —SOₘR_{ψ} (wherein R_{λ}, R_{π}, m and R_{ψ} are as defined earlier). Groups, such as ethenyl or vinyl (CH═CH₂), 1-propylene or allyl (—CH₂CH═CH₂), iso-propylene (—C(CH₃)═CH₂), bicyclo[2.2.1]heptene, and the like, exemplify this term.

The term “alkenylene” unless otherwise specified, refers to a diradical of a branched or unbranched unsaturated hydrocarbon group preferably having from 2 to 6 carbon atoms with cis, trans or geminal geometry. In the event that alkenylene is attached to the heteroatom, the double bond cannot be alpha to the heteroatom. The alkenylene group can be connected by two bonds to the rest of the structure of compound of Formula I. Alkenylene may further be substituted with one or more substituents such as alkyl, alkenyl, alkynyl, alkoxy, cycloalkyl, acyl, acylamino, acyloxy, —NHC(═O)R_{λ}, —NR_{λ}R_{π}, —C(═O)NR_{λ}R_{π}, —NHC(═O)NR_{λ}R_{π}, —OC(═O)NR_{λ}R_{π}, (wherein R_{λ} and R_{π} are the same as defined earlier), alkoxycarbonylamino, azido, cyano, halogen, hydroxy, oxo, thiocarbonyl, carboxy, —COOR_{ψ} (wherein R_{ψ} is the same as defined earlier), arylthio, thiol, alkylthio, aryl, aralkyl, aryloxy, heterocyclyl, heteroaryl, heterocyclyl alkyl, heteroaryl alkyl, aminosulfonyl, alkoxyamino, nitro, —S(O)ₘR_{ψ} (wherein nut and R_{ψ} are the same as defined earlier). Unless otherwise constrained by the definition, all substituents may optionally be further substituted by 1-3 substituents chosen from alkyl, alkenyl, alkynyl, carboxy, —COOR_{ψ} (wherein nit is the same as defined earlier), hydroxy, alkoxy, halogen, —CF₃, cyano, —NR_{λ}R_{π}, —C(═O)NR_{λ}R_{π}, —OC(═O)NR_{λ}R_{π}, (wherein R_{λ} and R_{π} are the same as defined earlier) and —S(O)ₘR_{ψ} (wherein R_{ψ} and m are the same as defined earlier).

The term “alkynyl,” unless otherwise specified, refers to a monoradical of an unsaturated hydrocarbon, having from 2 to 20 carbon atoms. Alkynyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkynyl groups are attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)R_{λ}, —NR_{λ}R_{π}, —NHC(═O)NR_{λ}R_{π}, —C(═O)NR_{λ}R_{π}, —O—C(═O)NR_{λ}R_{π}, or —SOₘR_{ψ} (wherein R_{λ}, R_{π}, m and R_{ψ} are the same as defined earlier). Unless otherwise constrained by the definition, alkynyl substituents optionally may be substituted further by 1-3 substituents selected from alkyl, alkenyl, alkynyl, carboxy, carboxyalkyl, hydroxy, alkoxy, halogen, CF₃, —NR_{λ}R_{π}, —C(═O)NR_{λ}R_{π}, —NHC(═O)NR_{λ}R_{π}, —C(═O)NR_{λ}R_{π}, cyano or —SOₘR_{ψ} (wherein R_{λ}, R_{π}, m and R_{ψ} are the same as defined earlier).

The term “alkynylene” unless otherwise specified, refers to a diradical of a triply-unsaturated hydrocarbon, preferably having from 2 to 6 carbon atoms. In the event that alkynylene is attached to the heteroatom, the triple bond cannot be alpha to the heteroatom. The alkenylene group can be connected by two bonds to the rest of the structure of compound of Formula I. Alkynylene may further be substituted with one or more substituents such as alkyl, alkenyl, alkoxy, cycloalkyl, acyl, acylamino, acyloxy, alkoxycarbonylamino, azido, cyano, halogen, hydroxy, oxo, thiocarbonyl, carboxy, arylthio, thiol, alkylthio, aryl, aralkyl, aryloxy, aminosulfonyl, nitro, heterocyclyl, heteroaryl, heterocyclyl alkyl, heteroarylalkyl, —NHC(═O)R_{λ}, —NR_{λ}R_{π}, —NHC(═O)NR_{λ}R_{π}, —C(═O)NR_{λ}R_{π}, —OC(═O)NR_{λ}R_{π} (wherein R_{λ} and R_{π} are the same as defined earlier), —S(O)ₘR_{ψ} (wherein n and m are the same as defined earlier). Unless otherwise constrained by the definition, all substituents may optionally be further substituted by 1-3 substituents chosen from alkyl, alkenyl, alkynyl, carboxy, —COOR_{ψ}, (wherein R_{ψ} is the same as defined earlier), hydroxy, alkoxy, halogen, CF₃, —NR_{λ}R_{π}, —C(═O)NR_{λ}R_{π}, —NHC(═O)NR_{λ}R_{π}, —C(═O)NR_{λ}R_{π} (wherein R_{λ} and R_{π} are the same as defined earlier), cyano, and —S(O)ₘR_{ψ} (wherein R_{ψ} and m are the same as defined earlier).

The term “alkoxy” denotes the group O-alkyl, wherein alkyl is the same as defined above.

The term “aryl,” unless otherwise specified, refers to aromatic system having 6 to 14 carbon atoms, wherein the ring system can be mono-, bi- or tricyclic and are carbocyclic aromatic groups. For example, aryl groups include, but are not limited to, phenyl, biphenyl, anthryl or napthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COOR_{ψ}, NHC(═O)R_{λ}, —NR_{λ}R_{π}, —C(═O)NR_{λ}R_{π}, —NHC(═O)NR_{λ}R_{π}, —O—C(═O)NR_{λ}R_{π}, —SOₘR_{ψ}, carboxy, heterocyclyl, heteroaryl, heterocyclylalkyl, heteroarylalkyl or amino carbonyl amino, mercapto, haloalkyl, optionally substituted aryl, optionally substituted heterocyclylalkyl, thioalkyl, —CONHR_{π}, —OCOR_{π}, —COR_{π}, —NHSO₂R_{π} or —SO₂NHR_{π} (wherein R_{λ}, R_{π}, m and R_{ψ} are the same as defined earlier). Aryl groups optionally may be fused with a cycloalkyl group, wherein the cycloalkyl group may optionally contain heteroatoms selected from O, N or S. Groups such as phenyl, naphthyl, anthryl, biphenyl, and the like exemplify this term.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl, propylphenyl, naphthylmethyl and the like.

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like or multiple ring structures, including adamantanyl, and bicyclo[2.2.1]heptane or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{λ}R_{π}, —NHC(═O)NR_{λ}R_{π}, —NHC(═O)R_{π}, —C(═O)NR_{λ}R_{π}, —O—C(═O)NR_{λ}R_{π}, nitro, heterocyclyl, heteroaryl, heterocyclylalkyl, heteroarylalkyl or SOARS (wherein R_{λ}, R_{π}, m and R_{ψ} are the same as defined earlier). Unless otherwise constrained by the definition, cycloalkyl substituents optionally may be substituted further by 1-3 substituents selected from alkyl, alkenyl, alkynyl, carboxy, hydroxy, alkoxy, halogen, CF₃, —NR_{λ}R_{π}, —C(═O)NR_{λ}R_{π}, —NHC(═O)NR_{λ}R_{π}, —OC(═O)NR_{λ}R_{π}, cyano or —SOₘR_{ψ} (wherein R_{π}, R_{π}, m and R_{ψ} are the same as defined earlier). “Cycloalkylalkyl” refers to alkyl-cycloalkyl group linked through alkyl portion, wherein the alkyl and cycloalkyl are the same as defined earlier.

“The term “aryloxy” denotes the group O-aryl, wherein aryl is as defined above.

The term “carboxy,” as defined herein, refers to —C(═O)OH.

The term “heteroaryl,” unless otherwise specified, refers to an aromatic ring structure containing 5 or 6 ring atoms or a bicyclic or tricyclic aromatic group having from 8 to 10 ring atoms, with one or more heteroatom(s) independently selected from N, O or S optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{λ}R_{π}, CH═NOH, —(CH₂)_{w}C(═O)R_{η} {wherein w is an integer from 0-4 and R_{η} is hydrogen, hydroxy, OR_{λ}, NR_{λ}R_{π}, —NHOR_{ω} or —NHOH}, —C(═O)NR_{λ}R_{π}, —NHC(═O)NR_{λ}R_{π}, —SOₘR_{ψ}, —O—C(═O)NR_{λ}R_{π}, —O—C(═O)R_{λ}, or —O—C(═O)OR_{λ} (wherein m, R_{ψ}, R_{λ} and R_{π} are as defined earlier and R_{ω} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzthiazinyl, benzthiazinonyl, benzoxazinyl, benzoxazinonyl, quinazonyl, carbazolyl phenothiazinyl, phenoxazinyl, benzothiazolyl or benzoxazolyl, and the like.

The term “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, optionally substituted aryl, alkoxy, alkaryl, cyano, nitro, oxo, carboxy, optionally substituted heterocyclyl, optionally substituted heterocyclylalkyl, optionally substituted heteroaryl, —O—C(═O)R_{λ}, —O—C(═O)OR_{λ}, —C(═O)NR_{λ}R_{π}, SOₘR_{ψ}, —O—C(═O)NR_{λ}R_{π}, —NHC(═O)NR_{λ}R_{π}, —NR_{λ}R_{π}, mercapto, haloalkyl, thioalkyl, —COOR_{ψ}, —COONHR_{λ}, —COR_{λ}, —NHSO₂R_{λ} or SO₂NHR_{λ} (wherein m, R_{λ}, R_{ψ} and R_{π} are as defined earlier) or guanidine. Heterocyclyl can optionally include rings having one or more double bonds. Such ring systems can be mono-, bi- or tricyclic. Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, benzoxazinyl, benzthiazinyl, imidazolyl, benzimidazolyl, tetrazolyl, carbaxolyl, indolyl, phenoxazinyl, phenothiazinyl, dihydropyridinyl, dihydroisoxazolyl, dihydrobenzofuryl, azabicyclohexyl, thiazolidinyl, dihydroindolyl, pyridinyl, isoindole 1,3-dione, piperidinyl, tetrahydropyranyl, piperazinyl, 3H-imidazo[4,5-b]pyridine, isoquinolinyl, 1H-pyrrolo[2,3-b]pyridine or piperazinyl and the like.

“Heteroarylalkyl” refers to alkyl-heteroaryl group linked through alkyl portion, wherein the alkyl and heteroaryl are as defined earlier.

“Heterocyclylalkyl” refers to alkyl-heterocyclyl group linked through alkyl portion, wherein the alkyl and heterocyclyl are as defined earlier.

Acyl” refers to —C(═O)R″ wherein R″ is selected from hydrogen, alkyl, cycloalkyl, aryl, aralkyl, heteroaryl, heterocyclyl, heteroarylalkyl or heterocyclylalkyl.

“Thiocarbonyl” refers to —C(═S)H. “Substituted thiocarbonyl” refers to —C(═S)R″, wherein R″ is selected from alkyl, cycloalkyl, aryl, aralkyl, heteroaryl, heterocyclyl, heteroarylalkyl or heterocyclylalkyl, amine or substituted amine.

The term “leaving group” refers to groups that exhibit or potentially exhibit the properties of being labile under the synthetic conditions and also, of being readily separated from synthetic products under defined conditions. Examples of leaving groups include, but are not limited to, halogen (e.g., F, Cl, Br, I), triflates, tosylate, mesylates, alkoxy, thioalkoxy, or hydroxy radicals and the like.

The term “protecting groups” refers to moieties that prevent chemical reaction at a location of a molecule intended to be left unaffected during chemical modification of such molecule. Unless otherwise specified, protecting groups may be used on groups, such as hydroxy, amino, or carboxy. Examples of protecting groups are found in T. W. Greene and P. G. M. Wuts, “Protective Groups in Organic Synthesis”, 2^{nd} Ed., John Wiley and Sons, New York, N.Y., which is incorporated herein by reference. The species of the carboxylic protecting groups, amino protecting groups or hydroxy protecting groups employed are not critical, as long as the derivatised moieties/moiety is/are stable to conditions of subsequent reactions and can be removed without disrupting the remainder of the molecule.

The term “pharmaceutically acceptable salts” refers to derivatives of compounds that can be modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like. Pharmaceutically acceptable salts may also be formed by complete derivatization of the amine moiety e,g. quaternary ammonium salts. The quaternary ammonium salts of the compound of Formula I can be prepared by reaction of compound of Formula I with Q-Z wherein (Q can be selected from alkyl, alkenyl, alkynyl, cycloalkyl, heterocyclyl, aryl, heteroaryl, aralkyl, heteroarylalkyl or heterocyclylalkyl and Z is an anion disclosed in International Journal of Pharmaceutics, 33 (1986), page 202, for example, but not limited to, tartarate, chloride, bromide, iodide, sulphate, phosphate, nitrate, carbonate, fumarate, glutamate, citrate, methanesulphonate, benzenesulphonate, maleate or succinate).

### DETAILED DESCRIPTION OF THE INVENTION

The compounds disclosed herein may be prepared by methods represented by reaction sequences, for example, as generally shown in Schemes I and II:

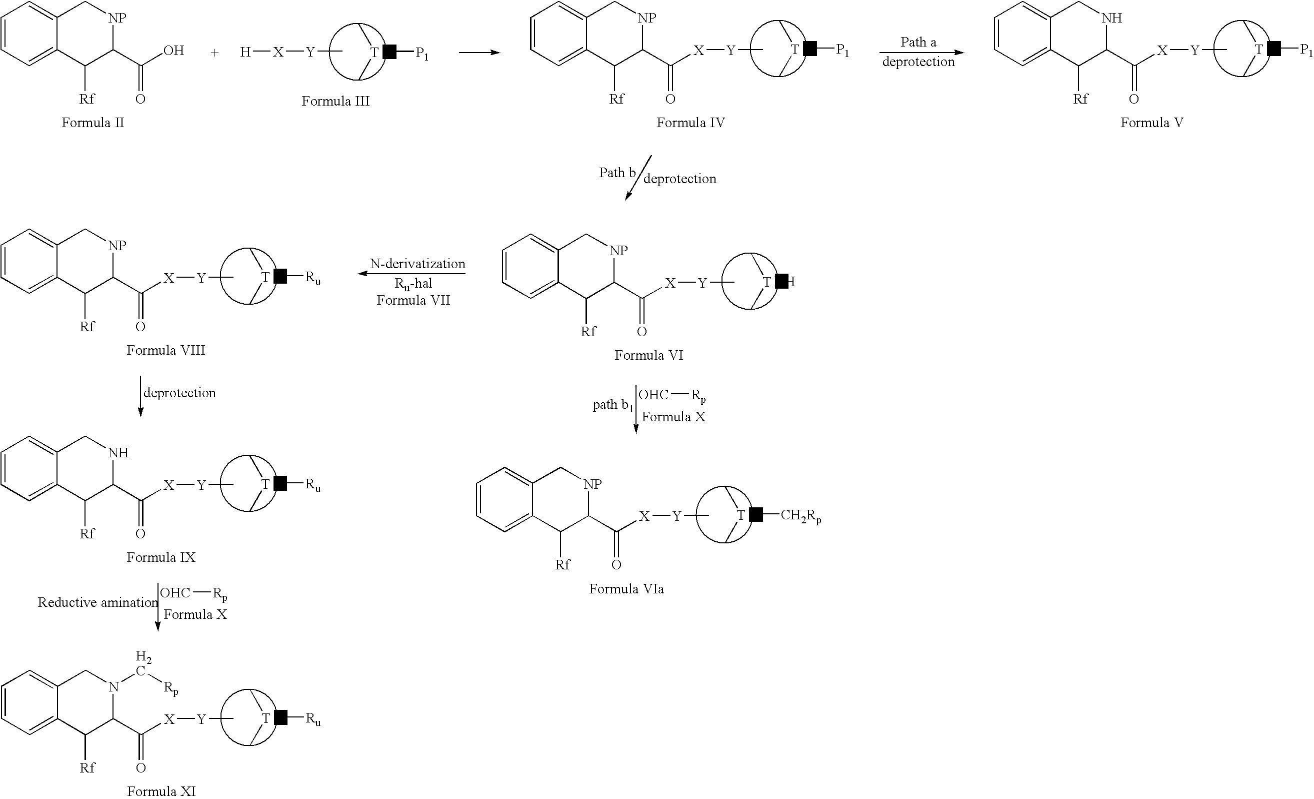

The compounds of Formulae IV, VI, VIa, VIII, IX, IX and XI can be prepared, for example, by following the procedure as described in Scheme I wherein,

### P is —C(═O)OC(CH₃)₃, —C(═O)OC(CH₃)₂CHBr₂ or C(═O)OC(CH₃)₂CCl₃;

P₁ is aralkyl;

### hal is Br, Cl or I; and

Rf, Rₚ, X, Y and Rᵤ are the same as defined earlier.

Condensation of a compound of Formula II with a compound of Formula III (when X is —O or —S; Y is the same as defined earlier) to give a compound of Formula IV can be carried out in an organic solvent (for example, dimethylformamide, tetrahydrofuran, diethylether or dioxane) with carbonyldiimidazole in the presence of a base (for example, sodium hydride, triethylamine, N-ethyldiisopropylamine or pyridine).

Alternatively, Condensation of a compound of Formula II with a compound of Formula III (when X is —O or —S; Y the same as defined earlier) can be carried out in an organic solvent (for example, toluene, heptane or xylene) in the presence of a base (for example, 1,8-diazabicyclo[5.4.0]undecen-7-ene or 1,4-diazabicyclo[2.2.2]octane).

Condensation of a compound of Formula II with a compound of Formula III (when X is —NH; Y is the same as defined earlier) to give a compound of Formula IV can be carried out in an organic solvent (for example, dimethylformamide, tetrahydrofuran, diethyl ether or dioxane) in the presence of a base (for example, N-methylmorpholine, triethylamine, diisopropylethylamine or pyridine) with a condensing agent (for example, 1-(3-dimethylaminopropyl)-3-ethylcarbodiimide hydrochloride (EDC.HCl) or dicyclohexylcarbodiimide).

The deprotection of a compound of Formula IV (path a, when P is —C(═O)OC(CH₃)₃ or —C(═O)OC(CH₃)₂CHBr₂) to give a compound of Formula V can be carried out in an acidic solution of an alcohol (for example, hydrochloric acid solution of methanol, ethanol, propanol, isopropylalcohol, ethylacetate or ether) or trifluoroacetic acid in dichloromethane.

The deprotection of a compound of Formula IV (path a, when P is —C(═O)OC(CH₃)₂CCl₃) to give a compound of Formula V can be carried out by a supernucleophile (for example, lithium cobalt (I) phthalocyanine, zinc and acetic acid or cobalt phthalocyanine).

The deprotection of a compound of Formula IV (path b, when P₁ is aralkyl) to give a compound of Formula VI can be carried out in an organic solvent (for example, methanol, ethanol, propanol or isopropylalcohol) in the presence of a deprotecting agent (for example, palladium on carbon in presence of hydrogen gas or palladium on carbon with a source of hydrogen gas (for example, ammonium formate solution, cyclohexene or formic acid)).

N-derivatization of a compound of Formula VI with a compound of Formula VII to give a compound of Formula VIII can be carried out in an organic solvent (for example, acetonitrile, dichloromethane, chloroform or carbon tetrachloride) in the presence of a base (for example, potassium carbonate, sodium carbonate or sodium bicarbonate).

The deprotection of a compound of Formula VIII (when P is —C(═O)OC(CH₃)₃ or —C(═O)OC(CH₃)₂CHBr₂) to give a compound of Formula IX can be carried out in an acidic solution of an alcohol (for example, hydrochloric acid solution of methanol, ethanol, propanol, isopropylalcohol, ethylacetate or ether) or trifluoroacetic acid in dichloromethane.

The deprotection of a compound of Formula VIII (when P is —C(═O)OC(CH₃)₂CCl₃) to give a compound of Formula IX can be carried out by a supernucleophile (for example, lithium cobalt (I) phthalocyanine, zinc and acetic acid or cobalt phthalocyanine).

The reductive amination of a compound of Formula IX with a compound of Formula X to give a compound of Formula XI can be carried out in an organic solvent selected from, dichloromethane, dichloroethane, chloroform or carbon tetrachloride with reducing agent selected from, sodium triacetoxyborohydride or sodium cyanoborohydride.

The reductive amination of a compound of Formula VI (path b1) with a compound of Formula X to give a compound of Formula VIa can be carried out in an organic solvent selected from, dichloromethane, dichloroethane, chloroform or carbon tetrachloride with reducing agent selected from, sodium triacetoxyborohydride or sodium cyanoborohydride.

Particular illustrative compounds which can be prepared following, for example, Scheme I include:
Tert-butyl 3-{[(3-benzyl-3-azabicyclo[3.1.0]hex-6-yl)amino]carbonyl}-3,4-dihydroisoquinoline-2(1H)-carboxylate (Compound No. 20),
Tert-butyl 3-({[(3-benzyl-3-azabicyclo[3.1.0]hex-6-yl)methyl]amino}carbonyl)-3,4-dihydroisoquinoline-2(1H)-carboxylate (Compound No. 25),
Tert-butyl 3-{[(3-benzyl-3-azabicyclo[3.2.1]oct-8-yl)amino]carbonyl}-3,4-dihydroisoquinoline-2(1H)-carboxylate (Compound No. 26).

Particular illustrative compounds which can be prepared following, for example, Scheme I path a include:
N-(3-benzyl-3-azabicyclo[3.2.1]oct-8-yl)-1,2,3,4-tetrahydroisoquinoline-3-carboxamide (Compound No. 1),
N-[(3-benzyl-3-azabicyclo[3.1.0]hex-6-yl)methyl]-1,2,3,4-tetrahydroisoquinoline-3-carboxamide (Compound No. 5),
(3-benzyl-3-azabicyclo[3.1.0]hex-6-yl)methyl 1,2,3,4-tetrahydroisoquinoline-3-carboxylate (Compound No. 6).

Particular illustrative compounds which can be prepared following, for example, Scheme I path b include:
N-{[3-(Morpholin-4-ylcarbonyl)-3-azabicyclo[3.1.0]hex-6-yl]methyl}-1,2,3,4-tetrahydroisoquinoline-3-carboxamide (Compound No. 9),
N-{[3-(Morpholin-4-ylcarbonyl)-3-azabicyclo[3.1.0]hex-6-yl]methyl}-2-propyl-1,2,3,4-tetrahydroisoquinoline-3-carboxamide (Compound No. 11),
Dihydrochloride salt of N-{3-[2-(2,3-dihydro-1-benzofuran-6-yl)ethyl]-3-azabicyclo[3.2.1]oct-8-yl}-1,2,3,4-tetrahydroisoquinoline-3-carboxamide (Compound No. 12),
Tert-butyl 3-[({3-[2-(2,3-dihydro-1-benzofuran-6-yl)ethyl]-3-azabicyclo[3.2.1]oct-8-yl}amino)carbonyl]-3,4-dihydroisoquinoline-2(1H)-carboxylate (Compound No. 13),
Dihydrochloride salt of N-[3-(1,3-benzodioxol-5-ylmethyl)-3-azabicyclo[3.2.1]oct-8-yl]-1,2,3,4-tetrahydroisoquinoline-3-carboxamide (Compound No. 14),
Dihydrochloride salt of N-{3-[2-(1,3-benzodioxol-5-yl)ethyl]-3-azabicyclo[3.2.1]oct-8-yl}-1,2,3,4-tetrahydroisoquinoline-3-carboxamide (Compound No. 15),
N-[3-(1,3-Benzodioxol-5-ylmethyl)-3-azabicyclo[3.2.1]oct-8-yl]-2-propyl-1,2,3,4-tetrahydroisoquinoline-3-carboxamide (Compound No. 16),
N-{3-[2-(1,3-Benzodioxol-5-yl)ethyl]-3-azabicyclo[3.2.1]oct-8-yl}-2-propyl-1,2,3,4-tetrahydroisoquinoline-3-carboxamide (Compound No. 17),
N-{3-[2-(2,3-Dihydro-1-benzofuran-6-yl)ethyl]-3-azabicyclo[3.2.1]oct-8-yl}-2-propyl-1,2,3,4-tetrahydroisoquinoline-3-carboxamide (Compound No. 18),
Tert-butyl 3-[(3-azabicyclo[3.2.1]oct-8-ylamino)carbonyl]-3,4-dihydroisoquinoline-2(1H)-carboxylate (Compound No. 19),
Tert-butyl 3-({[3-(1,3-benzodioxol-5-ylmethyl)-3-azabicyclo[3.2.1]oct-8-yl]amino}carbonyl)-3,4-dihydroisoquinoline-2(1H)-carboxylate (Compound No. 21),
Tert-butyl 3-[({3-[2-(1,3-benzodioxol-5-yl)ethyl]-3-azabicyclo[3.1.0]hex-6-yl}amino)carbonyl]-3,4-dihydroisoquinoline-2(1H)-carboxylate (Compound No. 22),
Tert-butyl 3-[({[3-(morpholin-4-ylcarbonyl)-3-azabicyclo[3.1.0]hex-6-yl]methyl}amino)carbonyl]-3,4-dihydroisoquinoline-2(1H)-carboxylate (Compound No. 23),
Tert-butyl 3-{[(3-azabicyclo[3.1.0]hex-6-ylmethyl)amino]carbonyl}-3,4-dihydroisoquinoline-2(1H)-carboxylate (Compound No. 24),
   and their pharmaceutically acceptable salts, pharmaceutically acceptable solvates, enantiomers, diastereomers, polymorphs or N-oxides.

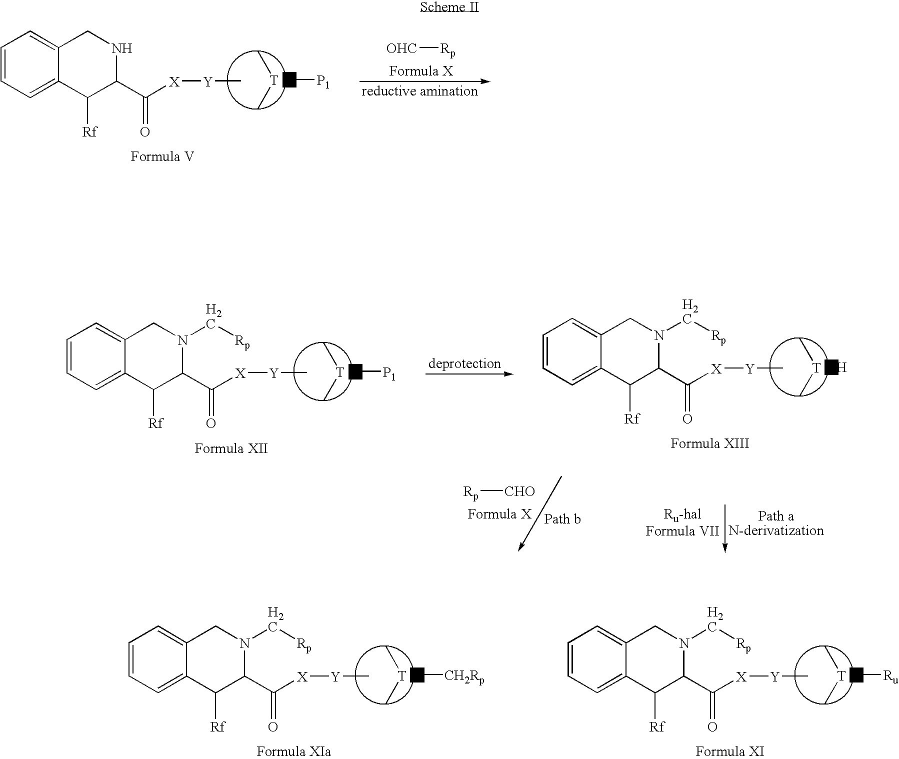

The compounds of Formulae XII, XIII, XI and XIa can be prepared, for example, by following the reaction procedure as depicted in Scheme II wherein, P₁, Rₚ, Rᵤ and hal are the same as defined earlier.

The reductive amination of a compound of Formula V with compound of Formula X to give a compound of Formula XII can be carried out in an organic solvent such as dichloromethane, dichloroethane, chloroform or carbon tetrachloride with reducing agent such as sodium triacetoxyborohydride or sodium cyanoborohydride.

The deprotection of a compound of Formula XII (when P₁ is -aralkyl) to give a compound of Formula XIII can be carried out in an organic solvent (for example, methanol, ethanol, propanol or isopropylalcohol) in the presence of a deprotecting agent (for example, palladium on carbon in presence of hydrogen gas or palladium on carbon with a source of hydrogen gas (for example, ammonium formate solution, cyclohexene or formic acid)).

The N-derivatization of a compound of Formula XIII (path a) with a compound of Formula VII to give a compound of Formula XI can be carried out in an organic solvent (for example, acetonitrile, dichloromethane, chloroform or carbon tetrachloride) in the presence of a base (for example, potassium carbonate, sodium carbonate or sodium bicarbonate).

The reductive amination of a compound of Formula XIII (path b) with a compound of Formula X to give a compound of Formula XIa can be carried out in an organic solvent selected from, dichloromethane, dichloroethane, chloroform or carbon tetrachloride with reducing agent selected from, sodium triacetoxyborohydride or sodium cyanoborohydride.

Particular illustrative compounds which can be prepared following, for example, Scheme II include:
N-(3-Azabicyclo[3.1.0]hex-6-ylmethyl)-2-propyl-1,2,3,4-tetrahydroisoquinoline-3-carboxamide (Compound No. 2),
N-[(3-Benzyl-3-azabicyclo[3.1.0]hex-6-yl)methyl]-2-propyl-1,2,3,4-tetrahydroisoquinoline-3-carboxamide (Compound No. 3),
N-(3-Benzyl-3-azabicyclo[3.2.1]oct-8-yl)-2-propyl-1,2,3,4-tetrahydroisoquinoline-3-carboxamide (Compound No. 4),
(3-Benzyl-3-azabicyclo[3.1.0]hex-6-yl)methyl 2-propyl-1,2,3,4-tetrahydroisoquinoline-3-carboxylate (Compound No. 7),
3-Azabicyclo[3.1.0]hex-6-ylmethyl 2-propyl-1,2,3,4-tetrahydroisoquinoline-3-carboxylate (Compound No. 8),
3-(Morpholin-4-ylcarbonyl)-3-azabicyclo[3.1.0]hex-6-yl]methyl 2-propyl-1,2,3,4-tetrahydroisoquinoline-3-carboxylate (Compound No. 10).
   and their pharmaceutically acceptable salts, pharmaceutically acceptable solvates, enantiomers, diastereomers, polymorphs or N-oxides.

In the above schemes, where specific bases, solvents, condensing agents, etc. are mentioned, it is to be understood that other acids, bases, solvents, condensing agents, hydrolyzing agents, etc, known to those skilled in an art may also be used. Similarly, the reaction temperature and duration of the reactions may be adjusted according to desired needs.

The compounds described herein can be produced and formulated as their racemic mixtures, enantiomers, diastereomers, rotamers, N-Oxides, polymorphs, solvates and pharmaceutically acceptable salts, as well as the active metabolites. Pharmaceutical compositions comprising the molecules of Formula I or metabolites, enantiomers, diastereomers, N-oxides, polymorphs, solvates or pharmaceutically acceptable salts thereof, in combination with pharmaceutically acceptable carrier and optionally included excipient can also be produced.

Where desired, the compounds of Formula I and/or their pharmaceutically acceptable salts, pharmaceutically acceptable solvates, stereoisomers, tautomers, racemates, prodrugs, metabolites, polymorphs or N-oxides may be advantageously used in combination with one or more other therapeutic agents. Examples of other therapeutic agents, which may be used in combination with compounds of Formula I of this invention and/or their pharmaceutically acceptable salts, pharmaceutically acceptable solvates, stereoisomers, tautomers, racemates, prodrugs, metabolites, polymorphs or N-oxides include, but are not limited to corticosteroids, beta agonists, leukotriene antagonists, 5-lipoxygenase inhibitors, anti-histamines, antitussives, dopamine receptor antagonists, chemokine inhibitors, p38 MAP Kinase inhibitors, and PDE-IV inhibitors.

The compositions can be administered by inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients. The compositions can be administered by the nasal respiratory route for local or systemic effect. Compositions can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered nasally from devices, which deliver the formulation in an appropriate manner.

Alternatively, compositions can be administered orally, rectally, parenterally (intravenously, intramuscularly or subcutaneously), intracistemally, intravaginally, intraperitoneally or topically.

Solid dosage forms for oral administration may be presented in discrete units, for example, capsules, cachets, lozenges, tablets, pills, powders, dragees or granules, each containing a predetermined amount of the active compound. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alignates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, and (i) lubricants, as for example, talc, calcium stearate, magnesium stearate, solid polyethylene glycols, sodium lauryl sulfate or mixtures thereof. In the case of capsules, tablets and pills, the dosage forms may also comprise buffering agents.

Solid compositions of a similar type may also be employed as fillers in soft and hard-filled gelatin capsules using such excipients as lactose or milk sugar as well as high molecular weight polyethylene glycols, and the like.

Solid dosage forms can be prepared with coatings and shells, such as enteric coatings and others well known in this art. They may contain opacifying agents, and can also be of such composition that they release the active compound or compounds in a certain part of the intestinal tract in a delayed manner. Examples of embedding compositions which can be used are polymeric substances and waxes.

The active compounds can also be in micro-encapsulated form, if appropriate, with one or more of the above mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

Besides such inert diluents, the composition can also include adjuvants, for example, wetting agents, emulsifying and suspending agents, sweetening, flavoring and perfuming agents, colorants or dyes.

Suspensions, in addition to the active compounds, may contain suspending agents, as for example, ethoxylated isostearyl alcohols, polyoxyethylene sorbitol and sorbitan esters, microcrystalline cellulose, aluminium metahydroxide, bentonite, agar-agar and tragacanth, or mixtures of these substances, and the like.

Dosage forms for topical administration of a compound of this invention include powder, spray, inhalant, ointment, creams, salve, jelly, lotion, paste, gel, aerosol, or oil. The active component is admixed under sterile conditions with a pharmaceutically acceptable carrier and any needed preservatives, buffers or propellants as may be required. Opthalmic formulations, eye ointments, powders and solutions are also contemplated as being within the scope of this invention.

Compositions suitable for parenteral injection may comprise pharmaceutically acceptable sterile aqueous or nonaqueous solutions, dispersions, suspensions or emulsions and sterile powders for reconstitution into sterile injectable solutions or dispersions. These preparations may contain anti-oxidants, buffers, bacteriostats and solutes, which render the compositions isotonic with the blood of the intended recipient. Aqueous and non-aqueous sterile suspensions may include suspending agents and thickening agents. The compositions may be presented in unit-dose or multi-dose containers, for example sealed ampoules and vials, and may be stored in a freeze-dried or lyophilized condition requiring only the addition of the sterile liquid carrier, for example, saline or water-for-injection immediately prior to use. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monosterate and gelatin.

Suppositories for rectal administration of the compound of Formula I can be prepared by mixing the drug with a suitable nonirritating excipient such as cocoa butter and polyethylene glycols or a suppository wax, which are solid at ordinary temperatures but liquid at body temperature and which therefore melt in the rectum or vaginal cavity and release the drug.

If desired, and for more effective distribution, the compounds can be incorporated into slow release or targeted delivery systems such as polymer matrices, liposomes, and microspheres. They may be sterilized, for example, by filtration through a bacteria-retaining filter, or by incorporating sterilizing agents in the form of sterile solid compositions, which can be dissolved in sterile water, or some other sterile injectable medium immediately before use.

Actual dosage levels of active ingredient in the compositions of the invention and spacing of individual dosages may be varied so as to obtain an amount of active ingredient that is effective to obtain a desired therapeutic response for a particular composition and method of administration. It will be understood, however, that the specific dose level for any particular patient will depend upon a variety of factors including the compound chosen, the body weight, general health, sex, diet, route of administration, the desired duration of treatment, rates of absorption and excretion, combination with other drugs and the severity of the particular disease being treated and is ultimately at the discretion of the physician.

The pharmaceutical compositions described herein can be produced and administered in dosage units, each unit containing a certain amount of at least one compound described herein and/or at least one physiologically acceptable addition salt thereof. The dosage may be varied over extremely wide limits as the compounds are effective at low dosage levels and relatively free of toxicity. The compounds may be administered in the low micromolar concentration, which is therapeutically effective, and the dosage may be increased as desired up to the maximum dosage tolerated by the patient.

The examples mentioned below demonstrate general synthetic procedures, as well as specific preparations of particular compounds. The examples are provided to illustrate particular details of the invention and do not limit the scope of the present invention.

### EXAMPLES

Various solvents, such as acetone, methanol, pyridine, ether, tetrahydrofuran, hexanes, and dichloromethane, were dried using various drying reagents according to procedures described in the literature. IR spectra were recorded as nujol mulls or a thin neat film on a Perkin Elmer Paragon instrument, Nuclear Magnetic Resonance (NMR) were recorded on a Varian XL-300 MHz or Bruker 400 MHz instrument using tetramethylsilane as an internal standard.

### Synthesis of 2-(tert-butoxycarbonyl)-1,2,3,4-tetrahydroisoquinoline-3-carboxylic acid (Formula II)

A mixture of the compound tetrahydroisoquinoline carboxylic acid (21.44 mmol), dioxane (40 ml), water (200 ml) and sodium hydroxide (2:1:1, 10 ml, 1 M) was cooled in an ice bath and stirred overnight. To the resulting reaction mixture was added di-tert-butoxy carbonyl anhydride (23.58 mmol) and stirred overnight. The mixture was concentrated under reduced pressure and subsequently cooled in an ice-bath. The residue thus obtained was diluted with ethylacetate and acidified with dilute potassium hydrogen sulphate solution to a pH of 2-3. The aqueous phase was extracted with ethylacetate, washed with water and brine, dried over anhydrous sodium sulphate, filtered and concentrated under reduced pressure to furnish the title compound.

¹H NMR (CDCl₃) δ: 7.25-7.14 (4H, m), 5.09-4.5 (2H, m), 4.47-4.42 (1H, m), 3.2-3.14 (2H, m), 1.50-1.40 (9H, m); Mass: 278 (M⁺+1); IR (cm⁻¹); 3425, 2977, 1700, 1406, 1165, 753.

### Synthesis of (3-benzyl-3-azabicyclo[3.1.0]hex-6-yl)methanol and 1-(3-benzyl-3-azabicyclo[3.1.0]hex-6-yl)methanamine (Formula III)

The title compound was prepared following the procedure as described in Synlett., (1996), 1097-1102.

### Synthesis of N-benzyl-6-amino-3-azabicyclo[3.1.0]hexane (Formula III)

The title compound was prepared following the procedure as described in Braish, T. F. et al., Syn. Lett., 1100 (1996).

### Synthesis of 3-benzyl-3-azabicyclo[3.2.1]octan-8-amine (Formula III)

### Step a: 3-Benzyl-3-azabicyclo[3.2.1]octan-8-one

To a mixture of the compound cyclopentanone (0.009 mol) in ethanol (20 ml) and water (5 ml) was added hydroxylamine hydrochloride (0.018 mol) followed by the addition of sodium bicarbonate (0.018 mol) portion wise. The reaction mixture was refluxed for 2-3 hours, which was subsequently concentrated under reduced pressure. The residue thus obtained was diluted with ethylacetate, washed with water and brine, dried over anhydrous sodium sulphate and concentrated under reduced pressure. The residue thus obtained was purified by column chromatography to furnish the title compound.

### Step b: 3-Benzyl-3-azabicyclo[3.2.1]octan-8-amine

To a solution of the compound obtained from step a above (0.0073 mol) in tetrahydrofuran (10 ml) was added a suspension of lithium aluminum hydride (0.0219 mol) in tetrahydrofuran (40 ml) and stirred for 6 hours followed by stirring for overnight. The reaction mixture was quenched with saturated sodium sulphate solution, which was subsequently filtered over celite pad and washed with ethylacetate. The organic layer was concentrated under reduced pressure to furnish the title compound.

¹HNMR (CDCl₃): δ 7.39-7.22 (5H, m), 3.51 (2H, s), 3.03-2.17 (4H, m), 1.91-1.47 (6H, m); Mass (m/z): 217;

### Synthesis of 1-(3-benzyl-3-azabicyclo[3.2.1]oct-8-yl)methanamine (Formula III)

### Step a: 3-benzyl-3-azabicyclo[3.2.1]octane-8-carbaldehyde oxime

To a solution of the compound 3-benzyl-3-azabicyclo[3.2.1]octane-8-carbaldehyde (0.25 g, 1.09 mmol) in ethanol (10 ml) was added sodium acetate (0.35 g, 4.25 mmol) and hydroxylamine hydrochloride (0.21 g, 3.065 mmol) and stirred the mixture at room temperature for 2 days. The reaction mixture was concentrated under reduced pressure and the residue thus obtained was diluted with saturated potassium carbonate solution. The mixture was extracted with ethylacetate, washed with water and brine, dried over anhydrous sodium sulphate and concentrated under reduced pressure to furnish the title compound. Yield: 0.2 g.

### Step b: 1-(3-benzyl-3-azabicyclo[3.2.1]oct-8-yl)methanamine

To a solution of the compound obtained from step a above (0.19 g, 0.778 mmol) in tetrahydrofuran (10 ml) was added a suspension of lithium aluminium hydride (0.147 g, 3.89 mmol) in tetrahydrofuran (20 ml) and refluxed for 16 hours. The reaction mixture was cooled in an ice bath and quenched with saturated sodium sulphate solution. The mixture was filtered, washed with water and brine dried over anhydrous sodium sulphate and concentrated under reduced pressure to furnish the title compound. Yield: 0.15 g

¹HNMR (CDCl₃): δ 7.34-7.19 (m, 5H), 3.49 (s, 2H), 2.74-2.69 (m, 2H), 2.49-2.46 (m, 2H), 2.09-2.00 (m, 4H, 1.59-1.25 (m, 5H).

### Scheme I, Procedure:

### General Procedure for Synthesis of Compound of Formula IV (Wherein X is —NH and Y is the Same as Defined Earlier)

To a solution of the acid of Formula II (1 mmol, 1 eq) and an amine of Formula III (1 mmol, 1 eq) in dimethylformamide (10 ml) was added hydroxybenzotriazole (1 mmol, 1 eq) and N-methylmorpholine (2 mmol, 2 eq) at 0° C. The resulting reaction mixture was stirred at 0° C. To the mixture was added 1-(3-dimethylaminopropyl)-3-ethylcarbodiimide hydrochloride (1 mmol, 1 eq) and stirred the mixture at 0° C. for 1 hour and then at room temperature for overnight. The mixture poured into quenched with sodium bicarbonate solution and extracted with ethylacetate. The ethylacetate layer was washed with water and brine, dried over anhydrous sodium sulphate and concentrated under reduced pressure. The residue thus obtained was purified by column chromatography to furnish the title compound.

Following compounds were prepared similarly,

### Tert-butyl 3-{[(3-benzyl-3-azabicyclo[3.1.0]hex-6-yl)amino]carbonyl}-3,4-dihydroisoquinoline-2(1H)-carboxylate (Compound No. 20)

¹HNMR (CDCl₃) δ: 7.27-7.18 (9H, m), 4.53-4.47 (3H, m), 3.50 (2H, s), 3.18-2.98 (5H, m), 2.30 (2H, s), 1.46 (9H, m), 1.25 (1H, m); Mass (m/z): 448 (M⁺+1); IR (cm¹); 3292, 1695, 1396, 1166, 744.

### Tert-butyl 3-({[(3-benzyl-3-azabicyclo[3.1.0]hex-6-yl)methyl]amino}carbonyl)-3,4-dihydroisoquinoline-2(1H)-carboxylate (Compound No. 25)

¹HNMR (CDCl₃) δ: 7.32-7.15 (9H, m), 4.64-4.51 (2H, m), 3.53 (2H, s), 3.22 (1H, m), 3.06-2.80 (4H, m), 2.23 (2H, m), 2.05 (1H, m), 1.46 (9H, m), 1.29-1.08 (4H, m); Mass (m/z): 462 (M⁺+1); IR (cm¹); 3330, 1664, 1166.

### Tert-butyl 3-{[(3-benzyl-3-azabicyclo[3.2.1]oct-8-yl)amino]carbonyl}-3,4-dihydroisoquinoline-2(1H)-carboxylate (Compound No. 26)

¹HNMR (CDCl₃) δ: 7.36-7.12 (9H, m), 3.60 (2H, m), 3.41 (6H, m), 2.62-2.55 (4H, m), 2.15-2.07 (4H, m), 1.37 (9H, m), 1.33 (2H, m); Mass (m/z): 476 (M⁺+1).

### General Procedure for Synthesis of Compound of Formula V

A mixture of the compound of Formula IV in methanolic hydrochloric acid (10-15 ml) was stirred at room temperature for 2-3 hours. The mixture was concentrated under reduced pressure and the residue thus obtained was diluted with water and basified with 10% aqueous sodium hydroxide. Aqueous layer was extracted with ethyl acetate, washed the organic layer with water and brine, dried over anhydrous sodium sulphate, filtered and concentrated under reduced pressure to furnish to furnish the title compound.

Following compounds were prepared similarly,

### N-(3-benzyl-3-azabicyclo[3.2.1]oct-8-yl)-1,2,3,4-tetrahydroisoquinoline-3-carboxamide (Compound No. 1)

¹H NMR (CDCl₃) δ: 7.66-7.08 (9H, m), 4.02 (2H, m), 3.97-3.43 (4H, m), 2.85 (1H, m), 2.35-2.26 (3H, m), 2.24-2.21 (5H, m), 1.30-1.28 (3H, m); Mass (m/z): 376 (M⁺+1).

### N-[(3-benzyl-3-azabicyclo[3.1.0]hex-6-yl)methyl]-1,2,34-tetrahydroisoquinoline-3-carboxamide (Compound No. 5)

¹H NMR (CDCl₃) δ: 7.31-7.04 (9H, m), 4.01 (1H, m), 3.58 (2H, s), 3.55 (2H, m), 3.22-2.96 (6H, m), 2.37 (1H, m), 1.30-1.12 (4H, m)

### (3-Benzyl-3-azabicyclo[3.1.0]hex-6-yl)methyl 1,2,3,4-tetrahydroisoquinoline-3-carboxylate (Compound No. 6)

¹HNMR (CDCl₃) δ: 7.29-7.02 (9H, m), 4.11-4.09 (2H, m), 3.98 (2H, m), 3.77 (1H, m), 3.59 (2H, s), 3.11-2.95 (4H, m), 2.35-2.33 (2H, m), 1.66 (1H, m), 1.65-1.25 (2H, m); Mass (m/z): 363 (M⁺+1).

### General Procedure for Synthesis of Compound of Formula VI (Wherein P₁ is Aralkyl)

To a solution of the compound of Formula IV (0.3125 mmol) in methanol (15 ml) was added palladium on carbon and anhydrous ammonium formate (1.8125 mmol) The mixture was refluxed for 1 hour and subsequently diluted with chloroform. The mixture was filtered over celite pad and washed with methanol. The filtrate was concentrated under reduced pressure and the residue thus obtained was diluted with water. The mixture was extracted with dichloromethane. The aqueous layer was basified with sodium hydroxide and extracted with ethylacetate. The organic layer was dried over anhydrous sodium sulphate and concentrated under reduced pressure to furnish the title compound.

Following compounds were prepared similarly,

### Tert-butyl 3-[(3-azabicyclo[3.2.1]oct-8-ylamino)carbonyl]-3,4-dihydroisoquinoline-2(1H)-carboxylate (Compound No. 19)

¹HNMR (CDCl₃) δ: 7.31-7.00 (4H, m), 4.55-4.35 (3H, m), 3.09-2.85 (5H, m), 2.23-2.21 (2H, m), 1.63-1.48 (10H, m); Mass (m/z): 358 (M⁺+1); IR (cm⁻¹); 3419, 1719.

### Tert-butyl 3-{[(3-azabicyclo[3.1.0]hex-6-ylmethyl)amino]carbonyl}-3,4-dihydroisoquinoline-2(1H)-carboxylate (Compound No. 24)

¹H NMR (CDCl₃) δ: 7.27-7.220 (4H, m), 4.62-4.50 (2H, m), 3.25-2.84 (6H, m), 2.44-1.94 (4H, m), 1.48-1.42 (9H, m), 1.34-1.05 (2H, m); Mass (m/z): 372 (M⁺+1).

### General Procedure for Synthesis of Compound of Formula VIII

To a mixture of the compound of Formula VI (0.8403 mmol) and the compound of Formula VII (0.8403 mmol) in acetonitrile was added potassium carbonate (1.6806 mmol) and potassium iodide (0.8403 mmol) and heated under reflux for overnight. The solvent was evaporated under reduced pressure and the residue thus obtained was diluted with water and ethylacetate. The organic layer was dried over anhydrous sodium sulphate, filtered and concentrated under reduced pressure. The residue thus obtained was purified by column chromatography to furnish the title compound.

Following compounds were prepared similarly,

### Tert-butyl 3-[({3-[2-(2,3-dihydro-1-benzofuran-6-yl)ethyl]-3-azabicyclo[3.2.1]oct-8-yl}amino)carbonyl]-3,4-dihydroisoquinoline-2(1H)-carboxylate (Compound No. 13)

¹HNMR (CDCl₃) δ: 7.31-7.19 (4H, m), 6.99 (1H, s), 6.88 (1H, d), 6.67 (1H, d), 4.55-4.50 (4H, m), 3.17-3.13 (4H, m), 2.22-2.18 (8H, m), 1.60-1.26 (13H, m); Mass (m/z): 504 (M⁺+1).

### Tert-butyl 3-({[3-(1,3-benzodioxol-5-ylmethyl)-3-azabicyclo[3.2.1]oct-8-yl]amino}carbonyl)-3,4-dihydroisoquinoline-2(1H)-carboxylate (Compound No. 21)

¹H NMR (CDCl₃) δ: 7.26-7.20 (4H, m), 6.72-6.64 (3H, m), 5.91 (2H, s), 4.42 (2H, m), 3.41 (2H, m), 2.99 (3H, m), 2.27 (2H, m), 1.57-1.47 (16H, m); Mass (m/z): 492 (M⁺+1); IR (cm⁻¹); 3380, 2361, 1593.

### Tert-butyl 3-[({3-[2-(1,3-benzodioxol-5-yl)ethyl]-3-azabicyclo[3.1.0]hex-6-yl}amino)carbonyl]-3,4-dihydroisoquinoline-2(1H)-carboxylate (Compound No. 22)

¹HNMR (CDCl₃) δ: 7.31-7.19 (4H, m), 6.70-6.58 (3H, m), 5.91 (2H, s), 3.63-3.10 (3H, m), 2.69-2.57 (5H, m), 2.18-2.17 (2H, m), 1.47-1.31 (11H, m); Mass (m/z): 502 (M⁺+1); IR (cm⁻¹); 3380, 2361, 1593.

### Tert-butyl 3-[({[3-(morpholin-4-ylcarbonyl)-3-azabicyclo[3.1.0]hex-6-yl]methyl}amino)carbonyl]-3,4-dihydroisoquinoline-2(1H)-carboxylate (Compound No. 23)

¹H NMR (CDCl₃) δ: 7.26-7.21 (4H, m), 3.68-3.62 (5H, m), 3.23-3.20 (10H, m), 1.59 (9H, m), 1.54 (7H, m); Mass (m/z): 485 (M⁺+1).

### General Procedure for Synthesis of Compound of Formula IX

Procedure I: To a solution of the compound of Formula VIII (0.2066 mmol) in dichloromethane (10-15 ml) was added trifluoroacetic acid and stirred the mixture for overnight. To the resulting reaction mixture was added aqueous sodium hydroxide (10%) and ethylacetate. The mixture was stirred for 1 hour. The organic layer was separated, washed with water and brine, dried over anhydrous sodium sulphate, filtered and concentrated under reduced pressure to furnish the title compound.

Procedure 2: A mixture of the compound of Formula VIII (0.2066 mmol) in methanolic hydrochloric acid (10-15 ml) was stirred at room temperature for 2-3 hours. The mixture was concentrated under reduced pressure and the residue thus obtained was diluted with water. Aqueous layer was extracted with ethyl acetate, washed the organic layer with water and brine, dried over anhydrous sodium sulphate, filtered and concentrated under reduced pressure to furnish to furnish dihydrochloride salt of the title compound.

A particular compound prepared following procedure I is:

### N-{[3-(Morpholin-4-ylcarbonyl)-3-azabicyclo[3.1.0]hex-6-yl]methyl}-1,2,3,4-tetrahydroisoquinoline-3-carboxamide (Compound No. 9)

¹H NMR (CDCl₃) δ: 7.33-7.05 (4H, m), 4.02 (2H, s), 3.80-3.79 (2H, m), 3.78-3.65 (6H, m), 3.31-3.21 (9H, m), 1.41-1.25 (3H, m); Mass (m/z): 385 (M⁺+1).

Particular compounds prepared following procedure II are:

### Dihydrochloride salt of N-{3-[2-(2,3-dihydro-1-benzofuran-6-yl)ethyl]-3-azabicyclo[3.2.1]oct-8-yl}-1,2,3,4-tetrahydroisoquinoline-3-carboxamide (Compound No. 12

¹HNMR (MeOD) δ: 7.32-7.26 (4H, m), 7.13 (1H, s), 6.98 (1H, d), 6.69 (1H, d), 4.54-4.43 (4H, m), 3.91-2.98 (13H, m), 2.20 (2H, m), 1.19 (1H, m); Mass (m/z): 404 (M⁺+1).

### Dihydrochloride salt of N-[3-(1,3-benzodioxol-5-ylmethyl)-3-azabicyclo[3.2.1]oct-8-yl]-1,2,3,4-tetrahydroisoquinoline-3-carboxamide (Compound No. 14)

¹HNMR (MeOD) δ: 7.31-7.29 (4H, m), 7.00-6.89 (3H, m), 6.02 (2H, s), 4.42 (2H, s), 4.29-4.19 (2H, m), 3.68-3.04 (8H, m), 2.12 (2H, m); Mass (m/z): 392 (M⁺+1); IR (cm⁻¹); 3424, 1679, 1448, 1253, 1034.

### Dihydrochloride salt of N-[3-[2-(1,3-benzodioxol-5-yl)ethyl]-3-azabicyclo[3.2.1]oct-8-yl]-1,2,3,4-tetrahydroisoquinoline-3-carboxamide (Compound No. 15)

¹HNMR (MeOD) δ: 7.31-7.28 (4H, m), 6.79-6.73 (3H, m), 5.92 (2H, s), 3.90 (2H, s), 3.88 (2H, m), 3.37-2.95 (10H, m), 2.11 (2H, m); Mass (m/z): 406 (M⁺+1).

### General Procedure for Synthesis of Compound of Formula XI

To a solution of the compound of Formula IX (1.385 mmol) in dichloroethane (10-15 ml) was added a compound of Formula X (1.385 mmol) and sodium triacetoxyborohydride (4.155 mmol) and stirred at room temperature for overnight. The reaction mixture was quenched with aqueous potassium hydroxide (5%) solution followed by the addition of ethylacetate and stirred the mixture at room temperature for 30 minutes. The organic layer was separated, washed with water and brine, dried over anhydrous sodium sulphate, filtered and concentrated under reduced pressure to furnish the title compound.

### N-{[3-(Morpholin-4-ylcarbonyl)-3-azabicyclo[3.1.0]hex-6-yl]methyl}-2-propyl-1,2,3,4-tetrahydroisoquinoline-3-carboxamide (Compound No. 11)

¹HNMR (CDCl₃) δ: 7.39-7.10 (4H, m), 3.87 (1H, m), 3.68-3.62 (8H, m), 3.23-3.04 (12H, m), 2.52-1.56 (2H, m), 1.54-1.26 (6H, m); Mass (m/z): 427 (M⁺+1).

### N-[3-(1,3-Benzodioxol-5-ylmethyl)-3-azabicyclo[3.2.1]oct-8-yl]-2-propyl-1,2,3,4-tetrahydroisoquinoline-3-carboxamide (Compound No. 16)

¹HNMR (MeOD) δ: 7.21-7.16 (3H, m), 7.08 (1H, m), 6.78-6.70 (3H, m), 5.92 (2H, s), 4.21 (1H, m), 3.85 (1H, m), 3.82 (1H, m), 3.64 (2H, m), 3.48-3.02 (9H, m), 2.39-2.35 (6H, m); Mass (m/z): 434 (M⁺+1).

### N-[3-[2-(1,3-Benzodioxol-5-yl)ethyl]-3-azabicyclo[3.2.1]oct-8-yl]-2-propyl-1,2,3,4-tetrahydroisoquinoline-3-carboxamide (Compound No. 17)

¹HNMR (MeOD) δ: 7.21-7.13 (3H, m), 7.09-7.07 (1H, m), 6.72-6.61 (3H, m), 5.91 (2H, s), 3.85 (1H, d), 3.64 (1H, d), 3.03 (1H, m), 3.01-2.64 (3H, m), 2.48 (1H, m), 2.47-2.45 (8H, m), 1.71-0.96 (8H, m); Mass (m/z): 448 (M⁺+1).

### N-{3-[2-(2,3-Dihydro-1-benzofuran-6-yl)ethyl]-3-azabicyclo[3.2.1]oct-8-yl}-2-propyl-1,2,3,4-tetrahydroisoquinoline-3-carboxamide (Compound No. 18)

¹HNMR (CDCl₃) δ: 7.26-6.69 (7H, m), 4.55-4.51 (2H, m), 3.85 (1H, m), 3.64 (1H, m), 3.32 (1H, m), 3.16-2.44 (10H, m), 1.53-0.91 (12H, m); Mass (m/z): 446 (M⁺+1).

### Scheme II:

### General Procedure for Synthesis of Compound of Formula XII

To a solution of the compound of Formula V (1.385 mmol) in dichloroethane (10-15 ml) was added a compound of Formula X (1.385 mmol) and stirred sodium triacetoxyborohydride (4.155 mmol) and stirred at room temperature for overnight. The reaction mixture was quenched with aqueous potassium hydroxide (5%) solution followed by the addition of ethylacetate and stirred the mixture at room temperature for 30 minutes. The organic layer was separated, washed with water and brine, dried over anhydrous sodium sulphate, filtered and concentrated under reduced pressure to furnish the title compound.

Following compounds were prepared similarly,

### N-[(3-Benzyl-3-azabicyclo[3.1.0]hex-6-yl)methyl]-2-propyl-1,2,3,4-tetrahydroisoquinoline-3-carboxamide (Compound No. 3)

¹HNMR (CDCl₃) δ: 7.31-7.08 (9H, m), 3.90 (1H, d), 3.67 (1H, d), 3.36 (2H, s), 3.05 (1H, m), 2.88 (4H, m), 2.85 (2H, m), 2.51 (2H, m), 2.29 (2H, m), 1.55-0.91 (8H, m); Mass (m/z): 404 (M⁺+1).

### N-(3-Benzyl-3-azabicyclo[3.2.1]oct-8-yl)-2-propyl-1,2,3,4-tetrahydroisoquinoline-3-carboxamide (Compound No. 4)

¹HNMR (CDCl₃) δ: 7.32-7.10 (9H, m), 3.38 (1H, m), 3.67 (2H, m), 3.08 (3H, m), 3.05 (2H, m), 2.59-2.57 (3H, m), 1.72-1.28 (13H, m); Mass (m/z): 418 (M⁺+1).

### (3-Benzyl-3-azabicyclo[3.1.0]hex-6-yl)methyl 2-propyl-1,2,3,4-tetrahydroisoquinoline-3-carboxylate (Compound No. 7)

¹HNMR (CDCl₃) δ: 7.31-7.01 (9H, m), 4.01 (1H, d), 3.92 (2H, d), 3.90 (1H, d), 3.87 (1H, m), 3.56 (2H, s), 2.92-2.31 (8H, m), 1.63-1.28 (8H, m); Mass (m/z): 405 (M⁺+1); IR (cm¹); 3442, 1732, 1165, 742.

### General Procedure for Synthesis of Compound of Formula XIII (Wherein P₁ is Aralkyl)

The title compound was prepared following the procedure as described for the synthesis of compound of Formula VI by using a compound of Formula XII in place compound of Formula IV.

Following compounds were prepared similarly,

### N-(3-Azabicyclo[3.1.0]hex-6-ylmethyl)-2-propyl-1,2,3,4-tetrahydroisoquinoline-3-carboxamide (Compound No. 2)

¹HNMR (CDCl₃) δ: 7.40-7.10 (4H, m), 3.90 (1H, d), 3.67 (1H, d), 3.06 (1H, m), 2.93 (4H, m), 2.91 (3H, m), 2.52 (2H, m), 1.56-1.23 (9H, m); Mass (m/z): 314 (M⁺+1).

### 3-Azabicyclo[3.1.0]hex-6-ylmethyl 2-propyl-1,2,3,4-tetrahydroisoquinoline-3-carboxylate (Compound No. 8)

¹HNMR (CDCl₃) δ: 7.26-7.02 (4H, m), 4.05-3.60 (5H, m), 2.92-2.34 (8H, m), 1.71-1.32 (8H, m); Mass (m/z): 315 (M⁺+1); IR (cm⁻¹); 3025, 1730, 1167, 744.

### General Procedure for Synthesis of Compound of Formula XI

To a mixture of the compound of Formula XIII (0.8403 mmol) and the compound of Formula VII (0.8403 mmol) in acetonitrile was added potassium carbonate (1.6806 mmol) and potassium bromide iodide (0.8403 mmol) and refluxed for overnight. The solvent was evaporated under reduced pressure and the residue thus obtained was diluted with water and ethylacetate. The organic layer was separated, washed with water and brine, dried over anhydrous sodium sulphate, filtered and concentrated under reduced pressure. The residue thus obtained was purified by column chromatography to furnish the title compound.

Following compound was prepared similarly,

### 3-(Morpholin-4-ylcarbonyl)-3-azabicyclo[3.1.0]hex-6-yl]methyl 2-propyl-1,2,3,4-tetrahydroisoquinoline-3-carboxylate (Compound No. 10)

¹HNMR (CDCl₃) δ: 7.26-7.01 (4H, m), 3.99-3.89 (5H, m), 3.69-3.20 (17H, m), 1.63-1.58 (3H, m); Mass (m/z): 428 (M⁺+1).

### Biological Activity

### Radioligand Binding Assays:

The affinity of test compounds for M₂ and M₃ muscarinic receptor subtypes were determined by [³H]-N-Methylscopolamine (NMS) binding studies using rat heart and submandibular gland respectively as described by Moriya et al., (Life Sci, 1999, 64(25): 2351-2358) with minor modifications. Specific binding of [³H]-NMS was also determined using membranes from Chinese hamster ovary (CHO) cells expressing cloned human muscarinic receptor subtypes.

### Membrane Preparation:

### (a) Rat Tissues

Submandibular glands and heart were isolated and placed in ice-cold homogenising buffer (HEPES 20 mM, 10 mM EDTA, pH 7.4) immediately after sacrifice. The tissues were homogenised in ten volumes of homogenising buffer and the homogenate was filtered through two layers of wet gauze and filtrate was centrifuged at 500 g for 10 min. The supernatant was subsequently centrifuged at 40,000 g for 20 min. The pellet thus obtained was resuspended in homogenising buffer (HEPES 20 mM, EDTA 10 mM, pH 7.4) and were stored at −70° C. until the time of assay.

### (b) Cho Cells Expressing Human Recombinant Receptors

The cell pellets were homogenised for 30 sec at 12,000 to 14,000 rpm, with intermittent gaps of 10-15 sec in ice-cold homogenising buffer (20 mM HEPES, 10 mM EDTA, pH 7.4). The homogenate was then centrifuged at 40,000 g for 20 min at 4° C. The pellet thus obtained was resuspended in homogenising buffer containing 10% sucrose and was stored at −70° C. until the time of assay.

### Ligand Binding Assay:

The compounds were dissolved and diluted in dimethyl sulphoxide. The membrane homogenates (5-10 μg protein) were incubated in 250 μL of assay buffer (20 mM HEPES, pH 7.4) at 24-25° C. for 3 hrs. Non-specific binding was determined in the presence of 1 μM Atropine. The incubation was terminated by vacuum filtration over GF/B fiber filter mats (Wallac) using Skatron cell harvester. The filters were then washed with ice-cold 50 mM Tris HCl buffer (pH 7.4). The filter mats were dried and transferred to 24 well plates (PET A No Cross Talk) followed by addition of 500 μl of scintillation cocktail. Radioactivity retained on filters was counted in Microbeta scintillation counter. The IC₅₀ & Kd were estimated by using the non-linear curve-fitting program using GraphPad Prism software. The value of inhibition constant, Ki was calculated from competitive binding studies by using Cheng & Prusoff's equation (Biochem Pharmacol, 1973, 22: 3099-3108), Ki=IC₅₀/(1+[L]/Kd), where [L] is the concentration of ligand [³H]-N-methyl scopolamine used in the particular experiment and Kd is the estimate of affinity of receptors to the ligand. The final result is expressed as the pKi value—the negative logarithm of Ki.

Compounds I-18 exhibited pKi in the range of 5.5 to 8.5 at rat/human muscarinic receptors receptors, alternatively expressed as Ki from about 1500 nM to about 6 nM, or, for example, from about 1000 nM to about 6 nM, or, for example, from about 500 nM to about 6 nM, or, for example, from about 250 nM to about 6 nM, or, for example, from about 100 nM to about 6 nM.

1. In-Vitro Functional Assay to Evaluate Efficacy of “MRA” in Combination with “PDE-IV Inhibitors”

### Animals and Anaesthesia:

Procure Guinea Pig (400-600 gm) and remove trachea under anesthesia (sodium pentobarbital, 300 mg/kg i.p) and immediately keep it in ice-cold Krebs Henseleit buffer. Indomethacin (10 uM) is present throughout the KH buffer to prevent the formation of bronchoactive prostanoids.

### Trachea Experiments:

Clean the tissue off adherent fascia and cut it into strips of equal size (with approx. 4-5 tracheal rings in each strip). Remove the epithelium by careful rubbing, minimizing damage to the smooth muscle. Open the trachea along the mid-dorsal surface with the smooth muscle band intact and make a series of transverse cuts from alternate sides so that they do not transect the preparation completely. Tie opposite end of the cut rings with the help of a thread. Mount the tissue in isolated tissue baths containing 10 ml Krebs Henseleit buffer maintained at 37° C. and bubbled with carbogen, at a basal tension of 1 gm. Change the buffer 4-5 times for about an hour. Equilibrate the tissue for 1 hr for stabilization. After 1 hr, challenge the tissue with 1 uM carbachol. Repeat this after every 2-3 washes till two similar consequetive responses are obtained. At the end of stabilization, wash the tissues for 30 minutes followed by incubation with suboptimal dose of MRA/Vehicle for 20 minutes prior to contraction of the tissues with 1 μM carbachol and subsequently assess the relaxant activity of the PDE-IV inhibitor [10⁻⁹ M to 10⁻⁴ M] on the stabilized developed tension/response. Record the contractile response of tissues either on Powerlab data acquisition system or on Grass polygraph (Model 7). Express the relaxation as percentage of maximum carbachol response. Express the data as mean ±s.e. mean for n observations. Calculate the EC₅₀ as the concentration producing 50% of the maximum relaxation to 1 μM carbachol. Compare percent relaxation between the treated and control tissues using non-parametric unpaired t-test. A p value of <0.05 is considered to be statistically significant.

2. In-Vivo Assay to Evaluate Efficacy of MRA in Combination with PDE-IV Inhibitors

### Drug Treatment

MRA (1 μg/kg to 1 mg/kg) and PDE-IV inhibitor (1 μg/kg to 1 mg/kg) are instilled intratracheally under anesthesia either alone or in combination.

### Method:

Male wistar rats weighing 200±20 gm are used in the study. Rats have free access to food and water. On the day of experiment, animals are exposed to lipopolysaccharide (LPS, 100 μg/ml) for 40 min. One group of vehicle treated rats is exposed to phosphate buffered saline (PBS) for 40 min. Two hours after LPS/PBS exposure, animals are placed inside a whole body plethysmograph (Buxco Electronics, USA) and exposed to PBS or increasing acetylcholine (1, 6, 12, 24, 48 and 96 mg/ml) aerosol until Penh values (index of airway resistance) of rats attained 2 times the value (PC-100) seen with PBS alone. The respiratory parameters are recorded online using Biosystem XA software, (Buxco Electronics, USA). Penh, at any chosen dose of acetylcholine is, expressed as percent of PBS response and the using a nonlinear regression analysis PC100 (2 folds of PBS value) values are computed. Percent inhibition is computed using the following formula.

$$\% Inhibition = \frac{PC {100}_{LPS} - PC {100}_{TEST}}{PC {100}_{LPS} - PC {100}_{PBS}} \times 100$$

### Where,

PC100_{LPS}=PC100 in untreated LPS challenged group
PC100_{TEST}=PC100 in group treated with a given dose of test compound
PC100_{PBS}=PC100 in group challenged with PBS

Immediately after the airway hyperreactivity response is recorded, animals are sacrificed and bronchoalveolar lavage (BAL) is performed. Collected lavage fluid is centrifuged at 3000 rpm for 5 min, at 4° C. Pellet is collected and resuspended in 1 ml HBSS. Total leukocyte count is performed in the resuspended sample. A portion of suspension is cytocentrifuged and stained with Leishmann's stain for differential leukocyte count. Total leukocyte and Neutrophil counts are expressed as cell count (millions cells ml⁻¹ of BAL). Percent inhibition is computed using the following formula.

$$\% Inhibition = \frac{{NC}_{LPS} - {NC}_{TEST}}{{NC}_{LPS} - {NC}_{CON}} \times 100$$

### Where,

NC_{LPS}=Percentage of neutrophil in untreated LPS challenged group
NC_{TEST}=Percentage of neutrophil in group treated with a given dose of test compound
NC_{CON}=Percentage of neutrophil in group not challenged with LPS

The percent inhibition data is used to compute ED₅₀ vales using Graph Pad Prism software (Graphpad Software Inc., USA).

3. In-Vivo Assay to Evaluate Efficacy of MRA in Combination with Corticosteroids

### Ovalbumin Induced Airway Inflammation:

Guinea pigs are sensitised on days 0, 7 and 14 with 50-μg ovalbumin and 10 mg aluminium hydroxide injected intraperitoneally. On days 19 and 20 guinea pigs are exposed to 0.1% w v⁻¹ ovalbumin or PBS for 10 min, and with 1% ovalbumin for 30 min on day 21. Guinea pigs are treated with test compound (0.1, 0.3 and 1 mg kg⁻¹) or standard 1 mg kg⁻¹ or vehicle once daily from day 19 and continued for 4 days. Ovalbumin/PBS challenge is performed 2 hours after different drug treatment.

24 hrs after the final ovalbumin challenge BAL is performed using Hank's balanced salt solution (HBSS). Collected lavage fluid is centrifuged at 3000 rpm for 5 min, at 4° C. Pellet is collected and resuspended in 1 ml HBSS. Total leukocyte count is performed in the resuspended sample. A portion of suspension is cytocentrifuged and stained with Leishmann's stain for differential leukocyte count. Total leukocyte and eosinophil count are expressed as cell count (millions cells ml⁻¹ of BAL). Eosinophil is also expressed as percent of total leukocyte count. % inhibition was computed using the following formula.

$$\% Inhibition = \frac{{Eos}_{OVA} - {Eos}_{TEST}}{{Eos}_{OVA} - {Eos}_{CON}} \times 100$$

### Where,

Eos_{OVA}=Percentage of eosinophil in untreated ovalbumin challenged group
Eos_{TEST}=Percentage of eosinophil in group treated with a given dose of test compound
Eos_{CON}=Percentage of eosinophil in group not challenged with ovalbumin.
4. In-Vivo Assay to Evaluate Efficacy of “MRA” in Combination with p38 Map Kinase Inhibitors

### Lipopolysaccharide (LPS) Induced Airway Hyperreactivity (AHR) and Neutrophilia:

### Drug Treatment:

MRA (1 μg/kg to 1 mg/kg) and p38 MAP kinase inhibitor (1 μg/kg to 1 mg/kg) are instilled intratracheally under anesthesia either alone or in combination.

### Method:

Male wistar rats weighing 200±20 gm are used in the study. Rats have free access to food and water. On the day of experiment, animals are exposed to lipopolysaccharide (LPS, 100 μg/ml) for 40 min. One group of vehicle treated rats is exposed to phosphate buffered saline (PBS) for 40 min. Two hours after LPS/PBS exposure, animals are placed inside a whole body plethysmograph (Buxco Electronics, USA) and exposed to PBS or increasing acetylcholine (1, 6, 12, 24, 48 and 96 mg/ml) aerosol until Penh values (index of airway resistance) of rats attained 2 times the value (PC-100) seen with PBS alone. The respiratory parameters are recorded online using Biosystem XA software, (Buxco Electronics, USA). Penh, at any chosen dose of acetylcholine is, expressed as percent of PBS response and the using a nonlinear regression analysis PC100 (2 folds of PBS value) values are computed. Percent inhibition is computed using the following formula.

$$\% Inhibition = \frac{PC {100}_{LPS} - PC {100}_{TEST}}{PC {100}_{LPS} - PC {100}_{PBS}} \times 100$$

### Where,

PC100_{LPS}=PC100 in untreated LPS challenged group
PC100_{TEST}=PC100 in group treated with a given dose of test compound
PC100_{PBS}=PC100 in group challenged with PBS

Immediately after the airway hyperreactivity response is recorded, animals are sacrificed and bronchoalveolar lavage (BAL) is performed. Collected lavage fluid is centrifuged at 3000 rpm for 5 min, at 4° C. Pellet is collected and resuspended in 1 ml HBSS. Total leukocyte count is performed in the resuspended sample. A portion of suspension is cytocentrifuged and stained with Leishmann's stain for differential leukocyte count. Total leukocyte and Neutrophil counts are expressed as cell count (millions cells ml⁻¹ of BAL). Percent inhibition is computed using the following formula.

$$\% Inhibition = \frac{{NC}_{LPS} - {NC}_{TEST}}{{NC}_{LPS} - {NC}_{CON}} \times 100$$

### Where,

NC_{LPS}=Percentage of neutrophil in untreated LPS challenged group
NC_{TEST}=Percentage of neutrophil in group treated with a given dose of test compound
NC_{CON}=Percentage of neutrophil in group not challenged with LPS
The percent inhibition data is used to compute ED₅₀ vales using Graph Pad Prism software (Graphpad Software Inc., USA).
5. In-Vivo Assay to Evaluate Efficacy of “MRA” in Combination with β2-Agonists

### Drug Treatment:

MRA (1 μg/kg to 1 mg/kg) and long acting β₂ agonist are instilled intratracheally under anesthesia either alone or in combination.

### Method

Wistar rats (250-350 gm) or balb/C mice (20-30 gm) are placed in body box of a whole body plethysmograph (Buxco Electronics., USA) to induce bronchoconstriction. Animals are allowed to acclimatise in the body box and are given successive challenges, each of 2 min duration, with PBS (vehicle for acetylcholine) or acetylcholine (i.e. 24, 48, 96, 144, 384, and 768 mg/ml). The respiratory parameters are recorded online using Biosystem XA software, (Buxco Electronics, USA) for 3 min. A gap of 2 min is allowed for the animals to recover and then challenged with the next higher dose of acetylcholine (ACh). This step is repeated until Penh of rats attained 2 times the value (PC-100) seen with PBS challenge. Following PBS/ACh challenge, Penh values (index of airway resistance) in each rat/mice is obtained in the presence of PBS and different doses of ACh. Penh, at any chosen dose of ACh is, expressed as percent of PBS response. The Penh values thus calculated are fed into Graph Pad Prism software (Graphpad Software Inc., USA) and using a nonlinear regression analysis PC100 (2 folds of PBS value) values are computed. % inhibition is computed using the following formula.

$$\% Inhibition = \frac{PC {100}_{TEST} - PC {100}_{CON}}{768 - PC {100}_{CON}} \times 100$$

### Where,

PC100_{CON}=PC100 in vehicle treated group
PC100_{TEST}=PC100 in group treated with a given dose of test compound
768=is the maximum amount of acetylcholine used.

We claim:

1. A compound of Formula I: 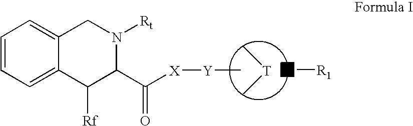
and their pharmaceutically acceptable salts, pharmaceutically acceptable solvates, enantiomers, diastereomers, polymorphs or N-oxides
wherein 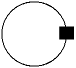
represents a nitrogen-containing ring having 5-8 ring atoms;
T is a bridging group selected from —(CH₂)ₙ—, —CH(Q)CH₂—, —CH₂CH(Q)CH₂—, —CH(Q)-, —CH₂—O—CH₂—, or —CH₂—NH—CH₂— (wherein the bridging group is attached to the two carbon atoms of the ring 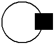
n is an integer selected from 0-3 (wherein when n is zero then T represents a direct bond);
Rf is hydrogen or Rₚ;
Y is alkylene or no atom (wherein when Y is no atom then X is directly attached to the ring 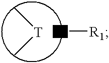
X is O, S or NRₛ (wherein Rₛ is as defined below);
R₁ is selected from hydrogen, aralkyl, halogen, —COOR₂ or Rᵤ (wherein Rᵤ is the same as defined below);
Rₜ is hydrogen, —COOR₂ or Rₚ (wherein Rₚ is alkyl, alkenyl, alkynyl, heterocyclylalkyl, heteroarylalkyl, aryl, aralkyl, heteroaryl, cycloalkyl or heterocyclyl);
Rᵤ is alkyl, aryl, heteroaryl, cycloalkyl, heterocyclyl, heteroarylalkyl, heterocyclylalkyl, —C(═O)NRₓR_{y}, —SO₂R₃, acyl (wherein R₃, Rₓ and R_{y} are the same as defined below);
Rₓ and R_{y} are independently selected from hydrogen, alkyl, cycloalkyl, aryl, halogen, aralkyl, heteroaryl, heterocyclyl, heteroarylalkyl or heterocyclylalkyl; Rₓ and R_{y} may also together join to form a heterocyclyl ring.
Rₛ is selected from hydrogen, alkyl, cycloalkyl, aryl, heteroaryl, heterocyclyl, heteroaryl, aralkyl, heteroarylalkyl or heterocyclylalkyl;
R₂ is independently selected from alkyl, alkenyl, alkynyl, aryl, aralkyl, heteroaryl, cycloalkyl, heterocyclyl, heterocyclylalkyl or heteroarylalkyl; and
R₃ is alkyl, aryl, heteroaryl, heterocyclyl, cycloalkyl, aralkyl, heteroarylalkyl, heterocyclylalkyl or —NRₓR_{y} (Rₓ and R_{y} are the same as defined earlier).

2. A compound selected from the group consisting of
N-(3-benzyl-3-azabicyclo[3.2.1]oct-8-yl)-1,2,3,4-tetrahydroisoquinoline-3-carboxamide (Compound No. 1),
N-(3-Azabicyclo[3.1.0]hex-6-ylmethyl)-2-propyl-1,2,3,4-tetrahydroisoquinoline-3-carboxamide (Compound No. 2),
N-[(3-Benzyl-3-azabicyclo[3.1.0]hex-6-yl)methyl]-2-propyl-1,2,3,4-tetrahydroisoquinoline-3-carboxamide (Compound No. 3),
N-(3-Benzyl-3-azabicyclo[3.2.1]oct-8-yl)-2-propyl-1,2,3,4-tetrahydroisoquinoline-3-carboxamide (Compound No. 4),
N-[(3-benzyl-3-azabicyclo[3.1.0]hex-6-yl)methyl]-1,2,3,4-tetrahydroisoquinoline-3-carboxamide (Compound No. 5),
(3-benzyl-3-azabicyclo[3.1.0]hex-6-yl)methyl 1,2,3,4-tetrahydroisoquinoline-3-carboxylate (Compound No. 6),
(3-Benzyl-3-azabicyclo[3.1.0]hex-6-yl)methyl 2-propyl-1,2,3,4-tetrahydroisoquinoline-3-carboxylate (Compound No. 7),
3-Azabicyclo[3.1.0]hex-6-ylmethyl 2-propyl-1,2,3,4-tetrahydroisoquinoline-3-carboxylate (Compound No. 8),
N-{[3-(Morpholin-4-ylcarbonyl)-3-azabicyclo[3.1.0]hex-6-yl]methyl}-1,2,3,4-tetrahydroisoquinoline-3-carboxamide (Compound No. 9),
3-(Morpholin-4-ylcarbonyl)-3-azabicyclo[3.1.0]hex-6-yl]methyl 2-propyl-1,2,3,4-tetrahydroisoquinoline-3-carboxylate (Compound No. 10),
N-{[3-(Morpholin-4-ylcarbonyl)-3-azabicyclo[3.1.0]hex-6-yl]methyl}-2-propyl-1,2,3,4-tetrahydroisoquinoline-3-carboxamide (Compound No. 11),
Dihydrochloride salt of N-{3-[2-(2,3-dihydro-1-benzofuran-6-yl)ethyl]-3-azabicyclo[3.2.1]oct-8-yl}-1,2,3,4-tetrahydroisoquinoline-3-carboxamide (Compound No. 12),
Tert-butyl 3-[({3-[2-(2,3-dihydro-1-benzofuran-6-yl)ethyl]-3-azabicyclo[3.2.1]oct-8-yl}amino)carbonyl]-3,4-dihydroisoquinoline-2(1H)-carboxylate (Compound No. 13),
Dihydrochloride salt of N-[3-(1,3-benzodioxol-5-ylmethyl)-3-azabicyclo[3.2.1]oct-8-yl]-1,2,3,4-tetrahydroisoquinoline-3-carboxamide (Compound No. 14),
Dihydrochloride salt of N-{3-[2-(1,3-benzodioxol-5-yl)ethyl]-3-azabicyclo[3.2.1]oct-8-yl}-1,2,3,4-tetrahydroisoquinoline-3-carboxamide (Compound No. 15),
N-[3-(1,3-Benzodioxol-5-ylmethyl)-3-azabicyclo[3.2.1]oct-8-yl]-2-propyl-1,2,3,4-tetrahydroisoquinoline-3-carboxamide (Compound No. 16),
N-{3-[2-(1,3-Benzodioxol-5-yl)ethyl]-3-azabicyclo[3.2.1]oct-8-yl}-2-propyl-1,2,3,4-tetrahydroisoquinoline-3-carboxamide (Compound No. 17),
N-{3-[2-(2,3-Dihydro-1-benzofuran-6-yl)ethyl]-3-azabicyclo[3.2.1]oct-8-yl}-2-propyl-1,2,3,4-tetrahydroisoquinoline-3-carboxamide (Compound No. 18),
Tert-butyl 3-[(3-azabicyclo[3.2.1]oct-8-ylamino)carbonyl]-3,4-dihydroisoquinoline-2(1H)-carboxylate (Compound No. 19),
Tert-butyl 3-{[(3-benzyl-3-azabicyclo[3.1.0]hex-6-yl)amino]carbonyl}-3,4-dihydroisoquinoline-2(1H)-carboxylate (Compound No. 20),
Tert-butyl 3-({[3-(1,3-benzodioxol-5-ylmethyl)-3-azabicyclo[3.2.1]oct-8-yl]amino}carbonyl)-3,4-dihydroisoquinoline-2(1H)-carboxylate (Compound No. 21),
Tert-butyl 3-[({3-[2-(1,3-benzodioxol-5-yl)ethyl]-3-azabicyclo[3.1.0]hex-6-yl}amino)carbonyl]-3,4-dihydroisoquinoline-2(1H)-carboxylate (Compound No. 22),
Tert-butyl 3-[({[3-(morpholin-4-ylcarbonyl)-3-azabicyclo[3.1.0]hex-6-yl]methyl}amino)carbonyl]-3,4-dihydroisoquinoline-2(1H)-carboxylate (Compound No. 23),
Tert-butyl 3-{[(3-azabicyclo[3.1.0]hex-6-ylmethyl)amino]carbonyl}-3,4-dihydroisoquinoline-2(1H)-carboxylate (Compound No. 24),
Tert-butyl 3-({[(3-benzyl-3-azabicyclo[3.1.0]hex-6-yl)methyl]amino}carbonyl)-3,4-dihydroisoquinoline-2(1H)-carboxylate (Compound No. 25),
Tert-butyl 3-{[(3-benzyl-3-azabicyclo[3.2.1]oct-8-yl)amino]carbonyl}-3,4-dihydroisoquinoline-2(1H)-carboxylate (Compound No. 26).

3. A pharmaceutical composition comprising a therapeutically effective amount of a compound as defined in claim 1 together with pharmaceutically acceptable carriers, excipients or diluents.

4. The use of compounds according to claim 1 for the manufacture of medicament for treating or preventing disease or disorder of the respiratory, urinary and gastrointestinal systems, wherein the disease or disorder is mediated through muscarinic receptors in mammal.

5. The use of compounds according to claim 1 for the manufacture of medicament for treating or preventing urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis in mammal.

6. The use of pharmaceutical composition according to claim 3 for the manufacture of medicament for treating or preventing disease or disorder of the respiratory, urinary and gastroinstestinal systems, wherein the disease or disorder is mediated through muscarinic receptors in mammal.

7. The use of pharmaceutical composition according to claim 3 for the manufacture of medicament for treating or preventing urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis in mammal.

8. A pharmaceutical composition comprising one or more compounds of Formula I 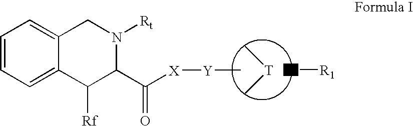
and their pharmaceutically acceptable salts, pharmaceutically acceptable solvates, enantiomers, diastereomers, polymorphs or N-oxides
wherein 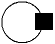
represents a nitrogen-containing ring having 5-8 ring atoms;
T is a bridging group selected from —(CH₂)ₙ—, —CH(Q)CH₂—, —CH₂CH(Q)CH₂—, —CH(Q)-, —CH₂—O—CH₂—, or —CH₂—NH—CH₂— (wherein the bridging group is attached to the two carbon atoms of the ring 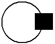
n is an integer selected from 0-3 (wherein when n is zero then T represents a direct bond);
Rf is hydrogen or Rₚ;
Y is alkylene or no atom (wherein when Y is no atom then X is directly attached to the ring 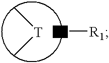
X is O, S or NRₛ (wherein Rₛ is as defined below);
R₁ is selected from hydrogen, aralkyl, halogen, —COOR₂ or Rᵤ (wherein Rᵤ is the same as defined below);
Rₜ is hydrogen, —COOR₂ or Rₚ (wherein Rₚ is alkyl, alkenyl, alkynyl, heterocyclylalkyl, heteroarylalkyl, aryl, aralkyl, heteroaryl, cycloalkyl or heterocyclyl);
Rᵤ is alkyl, aryl, heteroaryl, cycloalkyl, heterocyclyl, heteroarylalkyl, heterocyclylalkyl, —C(═O)NRₓR_{y}, —SO₂R₃, acyl (wherein R₃, Rₓ and R_{y} are the same as defined below);
Rₓ and R_{y} are independently selected from hydrogen, alkyl, cycloalkyl, aryl, halogen, aralkyl, heteroaryl, heterocyclyl, heteroarylalkyl or heterocyclylalkyl; Rₓ and R_{y} may also together join to form a heterocyclyl ring.
Rₛ is selected from hydrogen, alkyl, cycloalkyl, aryl, heteroaryl, heterocyclyl, heteroaryl, aralkyl, heteroarylalkyl or heterocyclylalkyl;
R₂ is independently selected from alkyl, alkenyl, alkynyl, aryl, aralkyl, heteroaryl, cycloalkyl, heterocyclyl, heterocyclylalkyl or heteroarylalkyl; and
R₃ is alkyl, aryl, heteroaryl, heterocyclyl, cycloalkyl, aralkyl, heteroarylalkyl, heterocyclylalkyl or —NRₓR_{y} (Rₓ and R_{y} are the same as defined earlier), and
at least one other active ingredients is selected from corticosteroids, beta agonists, leukotriene antagonists, 5-lipoxygenase inhibitors, anti-histamines, antitussives, dopamine receptor antagonists, chemokine inhibitors, p38 MAP Kinase inhibitors, and PDE-IV inhibitors.

9. A method of preparing a compound of Formula V and its pharmaceutically acceptable salts, pharmaceutically acceptable solvates, esters, enantiomers, diastereomers, N-oxides, polymorphs, prodrugs or metabolites, wherein the reaction comprises:
a. reacting a compound of Formula II 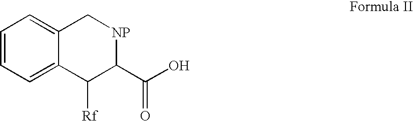
with a compound of Formula III 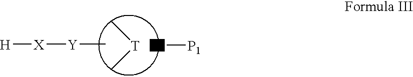
to give a compound of Formula IV; and 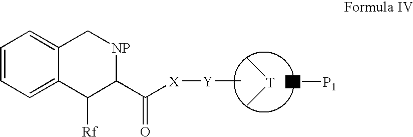
b. deprotecting a compound of Formula IV to give a compound of Formula V, 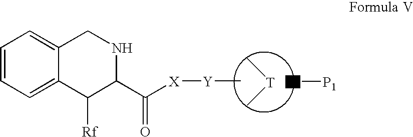
wherein
P is —C(═O)OC(CH₃)₃, —C(═O)OC(CH₃)₂CHBr₂ or C(═O)OC(CH₃)₂CCl₃;
P₁ is aralkyl; 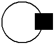
represents a nitrogen-containing ring having 5-8 ring atoms;
T is a bridging group selected from —(CH₂)ₙ—, —CH(Q)CH₂—, —CH₂CH(Q)CH₂—, —CH(Q)-, —CH₂—O—CH₂—, or —CH₂—NH—CH₂— (wherein the bridging group is attached to the two carbon atoms of the ring 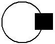
n is an integer selected from 0-3 (wherein when n is zero then T represents a direct bond);
Rf is hydrogen or Rₚ;
Rₚ is alkyl, alkenyl, alkynyl, heterocyclylalkyl, heteroarylalkyl, aryl, aralkyl, heteroaryl, cycloalkyl or heterocyclyl
Y is alkylene or no atom (wherein when Y is no atom then X is directly attached to the ring 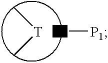
X is O, S or NRₛ (wherein Rₛ is as defined below); and
R₁ is selected from hydrogen, alkyl, cycloalkyl, aryl, heteroaryl, heterocyclyl, heteroaryl, aralkyl, heteroarylalkyl or heterocyclylalkyl.

10. A method of preparing a compound of Formula VIa and its pharmaceutically acceptable salts, pharmaceutically acceptable solvates, esters, enantiomers, diastereomers, N-oxides, polymorphs, prodrugs or metabolites, wherein the reaction comprises:
a. reacting a compound of Formula II 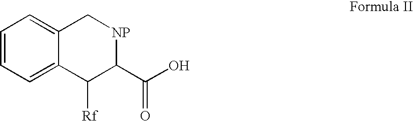
with a compound of Formula III 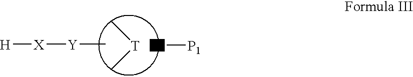
to give a compound of Formula IV; 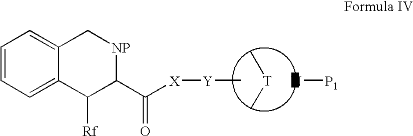
b. deprotecting a compound of Formula IV to give a compound of Formula VI; and 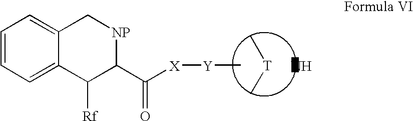
c. reacting a compound of Formula VI with a compound of Formula X
OHC—Rₚ Formula X
to give a compound of Formula VIa 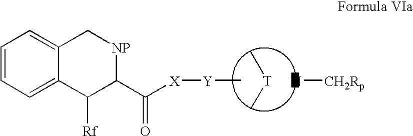
wherein
P is —C(═O)OC(CH₃)₃, —C(═O)OC(CH₃)₂CHBr₂ or C(═O)OC(CH₃)₂CCl₃;
P₁ is aralkyl; 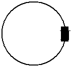
represents a nitrogen-containing ring having 5-8 ring atoms;
T is a bridging group selected from —(CH₂)ₙ—, —CH(Q)CH₂—, —CH₂CH(Q)CH₂—, —CH(Q)-, —CH₂—O—CH₂—, or —CH₂—NH—CH₂— (wherein the bridging group is attached to the two carbon atoms of the ring 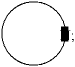
n is an integer selected from 0-3 (wherein when n is zero then T represents a direct bond);
Rf is hydrogen or Rₚ;
Rₚ is alkyl, alkenyl, alkynyl, heterocyclylalkyl, heteroarylalkyl, aryl, aralkyl, heteroaryl, cycloalkyl or heterocyclyl;
Y is alkylene or no atom (wherein when Y is no atom then X is directly attached to the ring 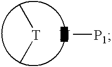
X is O, S or NRₛ (wherein Rₛ is as defined below); and
Rₛ is selected from hydrogen, alkyl, cycloalkyl, aryl, heteroaryl, heterocyclyl, heteroaryl, aralkyl, heteroarylalkyl or heterocyclylalkyl.

11. A method of preparing a compound of Formula XI and its pharmaceutically acceptable salts, pharmaceutically acceptable solvates, esters, enantiomers, diastereomers, N-oxides, polymorphs, prodrugs or metabolites, wherein the reaction comprises:
a. reacting a compound of Formula II 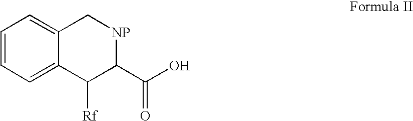
with a compound of Formula III 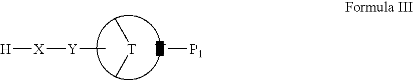
to give a compound of Formula IV; 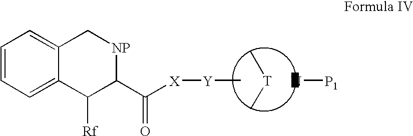
b. deprotecting a compound of Formula IV to give a compound of Formula VI; 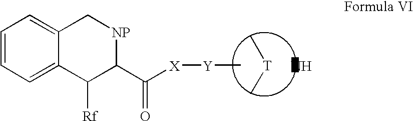
c. reacting a compound of Formula VI with a compound of Formula VII
Rᵤ-hal Formula VII
to give a compound of Formula VIII; 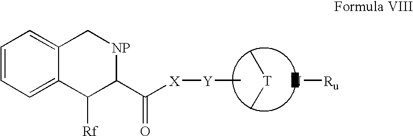
d. deprotecting a compound of Formula VIII to give a compound of Formula IX; and 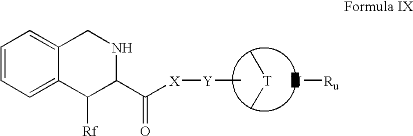
e. reacting a compound of Formula IX with a compound of Formula of X
OHC—Rₚ Formula X
to give a compound of Formula XI, 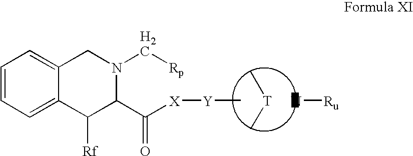
wherein
P is —C(═O)OC(CH₃)₃, —C(═O)OC(CH₃)₂CHBr₂ or C(═O)OC(CH₃)₂CCl₃;
P₁ is aralkyl;
hal is Br, Cl or I; 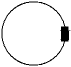
represents a nitrogen-containing ring having 5-8 ring atoms;
T is a bridging group selected from —(CH₂)ₙ—, —CH(Q)CH₂—, —CH₂CH(Q)CH₂—, —CH(Q)-, —CH₂—O—CH₂—, or —CH₂—NH—CH₂— (wherein the bridging group is attached to the two carbon atoms of the ring 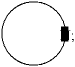
n is an integer selected from 0-3 (wherein when n is zero then T represents a direct bond);
Rf is hydrogen or Rₚ;
Rₚ is alkyl, alkenyl, alkynyl, heterocyclylalkyl, heteroarylalkyl, aryl, aralkyl, heteroaryl, cycloalkyl or heterocyclyl;
Y is alkylene or no atom (wherein when Y is no atom then X is directly attached to the ring 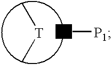
X is O, S or NRₛ (wherein Rₛ is as defined below);
Rₛ is selected from hydrogen, alkyl, cycloalkyl, aryl, heteroaryl, heterocyclyl, heteroaryl, aralkyl, heteroarylalkyl or heterocyclylalkyl;
Rᵤ is alkyl, aryl, heteroaryl, cycloalkyl, heterocyclyl, heteroarylalkyl, heterocyclylalkyl, —C(═O)NRₓR_{y}, —SO₂R₃, acyl (wherein R₃, Rₓ and R_{y} are the same as defined below);
Rₓ and R_{y} are independently selected from hydrogen, alkyl, cycloalkyl, aryl, halogen, aralkyl, heteroaryl, heterocyclyl, heteroarylalkyl or heterocyclylalkyl; Rₓ and R_{y} may also together join to form a heterocyclyl ring; and
R₃ is alkyl, aryl, heteroaryl, heterocyclyl, cycloalkyl, aralkyl, heteroarylalkyl, heterocyclylalkyl or —NRₓR_{y} (Rₓ and R_{y} are the same as defined earlier).

12. A method of preparing a compound of Formula XI and its pharmaceutically acceptable salts, pharmaceutically acceptable solvates, esters, enantiomers, diastereomers, N-oxides, polymorphs, prodrugs or metabolites, wherein the reaction comprises:
a. reacting a compound of Formula V 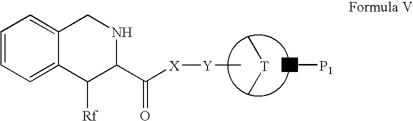
with a compound of Formula X
OHC—Rₚ Formula X
to give a compound of Formula XII; 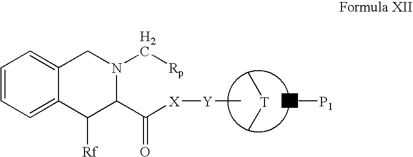
b. deprotecting a compound of Formula XII to give a compound of Formula XIII; and 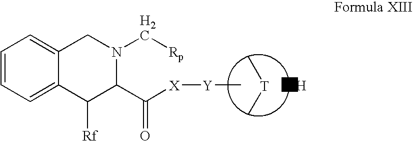
c. reacting a compound of Formula XIII with a compound of Formula VII
Rᵤ-hal Formula VII
to give a compound of Formula XI, 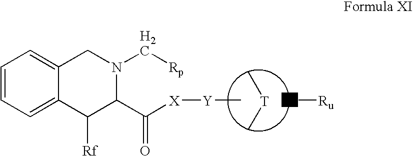
wherein
P is —C(═O)OC(CH₃)₃, —C(═O)OC(CH₃)₂CHBr₂ or C(═O)OC(CH₃)₂CCl₃;
P₁ is aralkyl;
hal is Br, Cl or I; 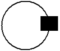
represents a nitrogen-containing ring having 5-8 ring atoms;
T is a bridging group selected from —(CH₂)ₙ—, —CH(Q)CH₂—, —CH₂CH(Q)CH₂—, —CH(Q)-, —CH₂—O—CH₂—, or —CH₂—NH—CH₂— (wherein the bridging group is attached to the two carbon atoms of the ring 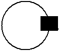
n is an integer selected from 0-3 (wherein when n is zero then T represents a direct bond);
Rf is hydrogen or Rₚ;
Rₚ is alkyl, alkenyl, alkynyl, heterocyclylalkyl, heteroarylalkyl, aryl, aralkyl, heteroaryl, cycloalkyl or heterocyclyl;
Y is alkylene or no atom (wherein when Y is no atom then X is directly attached to the ring 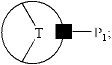
X is O, S or NRₛ (wherein Rₛ is as defined below);
Rₛ is selected from hydrogen, alkyl, cycloalkyl, aryl, heteroaryl, heterocyclyl, heteroaryl, aralkyl, heteroarylalkyl or heterocyclylalkyl;
Rᵤ is alkyl, aryl, heteroaryl, cycloalkyl, heterocyclyl, heteroarylalkyl, heterocyclylalkyl, —C(═O)NRₓR_{y}, —SO₂R₃, acyl (wherein R₃, Rₓ and R_{y} are the same as defined below);
Rₓ and R_{y} are independently selected from hydrogen, alkyl, cycloalkyl, aryl, halogen, aralkyl, heteroaryl, heterocyclyl, heteroarylalkyl or heterocyclylalkyl; Rₓ and R_{y} may also together join to form a heterocyclyl ring; and
R₃ is alkyl, aryl, heteroaryl, heterocyclyl, cycloalkyl, aralkyl, heteroarylalkyl, heterocyclylalkyl or —NRₓR_{y} (Rₓ and R_{y} are the same as defined earlier).

13. A method of preparing a compound of Formula XIa and its pharmaceutically acceptable salts, pharmaceutically acceptable solvates, esters, enantiomers, diastereomers, N-oxides, polymorphs, prodrugs or metabolites, wherein the reaction comprises:
a. reacting a compound of Formula V 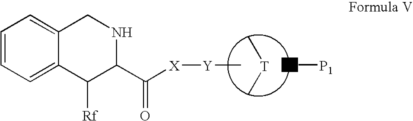
with a compound of Formula X
OHC—Rₚ Formula X
to give a compound of Formula XII; 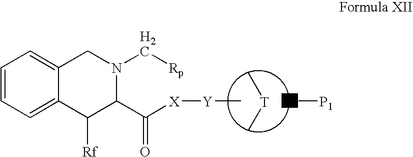
b. deprotecting a compound of Formula XII to give a compound of Formula XIII; and 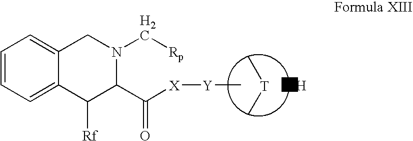
c. reacting a compound of Formula XIII with a compound of Formula X
Rₚ—CHO Formula X
to give a compound of Formula XIa, 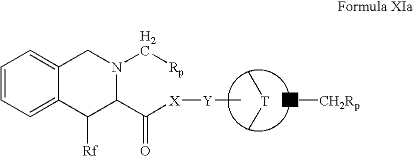
wherein
P is —C(═O)OC(CH₃)₃, —C(═O)OC(CH₃)₂CHBr₂ or —C(═O)OC(CH₃)₂CCl₃;
P₁ is aralkyl; 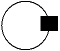
represents a nitrogen-containing ring having 5-8 ring atoms;
T is a bridging group selected from —(CH₂)ₙ—, —CH(Q)CH₂—, —CH₂CH(Q)CH₂—, —CH(Q)-, —CH₂—O—CH₂—, or —CH₂—NH—CH₂— (wherein the bridging group is attached to the two carbon atoms of the ring 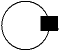
n is an integer selected from 0-3 (wherein when n is zero then T represents a direct bond);
Rf is hydrogen or Rₚ;
Rₚ is alkyl, alkenyl, alkynyl, heterocyclylalkyl, heteroarylalkyl, aryl, aralkyl, heteroaryl, cycloalkyl or heterocyclyl;
Y is alkylene or no atom (wherein when Y is no atom then X is directly attached to the ring 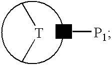
X is O, S or NRₛ (wherein Rₛ is as defined below); and
Rₛ is selected from hydrogen, alkyl, cycloalkyl, aryl, heteroaryl, heterocyclyl, heteroaryl, aralkyl, heteroarylalkyl or heterocyclylalkyl.
